SEQUENCE LISTING (209 sequences)

SEQ ID NO: 1 - protein, 36 aa, Homo sapiens
DHYNCVSSGGQCLYSACPIFTKIQGTCYRGKAKCCK

SEQ ID NO: 2 - protein, 33 aa, Artificial sequence
  Cleavage product of defensin
DHYNCVSSGGQCLYSACPIFTKIQGTCYRGKAK

SEQ ID NO: 3 - protein, 33 aa, Artificial sequence
  Cleavage product of defensin
NCVSSGGQCLYSACPIFTKIQGTCYRGKAKCCK

SEQ ID NO: 4 - protein, 31 aa, Artificial sequence
  Cleavage product of defensin
DHYNCVSSGGQCLYSACPIFTKIQGTCYRGK

SEQ ID NO: 5 - protein, 29 aa, Artificial sequence
  Cleavage product of defensin
DHYNCVSSGGQCLYSACPIFTKIQGTCYR

SEQ ID NO: 6 - protein, 28 aa, Artificial sequence
  Cleavage product of defensin
DHYNCVSSGGQCLYSACPIFTKIQGTCY

SEQ ID NO: 7 - protein, 28 aa, Artificial sequence
  Cleavage product of defensin
NCVSSGGQCLYSACPIFTKIQGTCYRGK

SEQ ID NO: 8 - protein, 26 aa, Artificial sequence
  Cleavage product of defensin
NCVSSGGQCLYSACPIFTKIQGTCYR

SEQ ID NO: 9 - protein, 25 aa, Artificial sequence
  Cleavage product of defensin
NCVSSGGQCLYSACPIFTKIQGTCY

SEQ ID NO: 10 - protein, 23 aa, Artificial sequence
  Cleavage product of defensin
YSACPIFTKIQGTCYRGKAKCCK

SEQ ID NO: 11 - protein, 22 aa, Artificial sequence
  Cleavage product of defensin
DHYNCVSSGGQCLYSACPIFTK

SEQ ID NO: 12 - protein, 22 aa, Artificial sequence
  Cleavage product of defensin
SACPIFTKIQGTCYRGKAKCCK

SEQ ID NO: 13 - protein, 20 aa, Artificial sequence
  Cleavage product of defensin
DHYNCVSSGGQCLYSACPIF

SEQ ID NO: 14 - protein, 19 aa, Artificial sequence
  Cleavage product of defensin
SACPIFTKIQGTCYRGKAK

SEQ ID NO: 15 - protein, 19 aa, Artificial sequence
  Cleavage product of defensin
NCVSSGGQCLYSACPIFTK

SEQ ID NO: 16 - protein, 17 aa, Artificial sequence
  Cleavage product of defensin
SACPIFTKIQGTCYRGK

SEQ ID NO: 17 - protein, 16 aa, Artificial sequence
  Cleavage product of defensin
TKIQGTCYRGKAKCCK

SEQ ID NO: 18 - protein, 17 aa, Artificial sequence
  Cleavage product of defensin
NCVSSGGQCLYSACPIF

SEQ ID NO: 19 - protein, 16 aa, Artificial sequence
  Cleavage product of defensin
TKIQGTCYRGKAKCCK

SEQ ID NO: 20 - protein, 15 aa, Artificial sequence
  Cleavage product of defensin
SACPIFTKIQGTCYR

SEQ ID NO: 21 - protein, 15 aa, Artificial sequence
  Cleavage product of defensin
YSACPIFTKIQGTCY

SEQ ID NO: 22 - protein, 14 aa, Artificial sequence
  Cleavage product of defensin
IQGTCYRGKAKCCK

SEQ ID NO: 23 - protein, 14 aa, Artificial sequence
  Cleavage product of defensin
DHYNCVSSGGQCLY

SEQ ID NO: 24 - protein, 14 aa, Artificial sequence
  Cleavage product of defensin
IQGTCYRGKAKCCK

SEQ ID NO: 25 - protein, 14 aa, Artificial sequence
  Cleavage product of defensin
SACPIFTKIQGTCY

SEQ ID NO: 26 - protein, 13 aa, Artificial sequence
  Cleavage product of defensin
DHYNCVSSGGQCL

SEQ ID NO: 27 - protein, 11 aa, Artificial sequence
  Cleavage product of defensin
NCVSSGGQCLY

SEQ ID NO: 28 - protein, 13 aa, Artificial sequence
  Cleavage product of defensin
TKIQGTCYRGKAK

SEQ ID NO: 29 - protein, 11 aa, Artificial sequence
  Cleavage product of defensin
TKIQGTCYRGK

SEQ ID NO: 30 - protein, 11 aa, Artificial sequence
  Cleavage product of defensin
IQGTCYRGKAK

SEQ ID NO: 31 - protein, 11 aa, Artificial sequence
  Cleavage product of defensin
NCVSSGGQCLY

SEQ ID NO: 32 - protein, 10 aa, Artificial sequence
  Cleavage product of defensin
NCVSSGGQCL

SEQ ID NO: 33 - protein, 9 aa, Artificial sequence
  Cleavage product of defensin
TKIQGTCYR

SEQ ID NO: 34 - protein, 9 aa, Artificial sequence
  Cleavage product of defensin
IQGTCYRGK

SEQ ID NO: 35 - protein, 8 aa, Artificial sequence
  Cleavage product of defensin
TKIQGTCY

SEQ ID NO: 36 - protein, 8 aa, Artificial sequence
  Cleavage product of defensin
RGKAKCCK

SEQ ID NO: 37 - protein, 8 aa, Artificial sequence
  Cleavage product of defensin
SACPIFTK

SEQ ID NO: 38 - protein, 7 aa, Artificial sequence
  Cleavage product of defensin
IQGTCYR

SEQ ID NO: 39 - protein, 7 aa, Artificial sequence
  Cleavage product of defensin
GKAKCCK

SEQ ID NO: 40 - protein, 6 aa, Artificial sequence
  Cleavage product of defensin
IQGTCY

SEQ ID NO: 41 - protein, 7 aa, Artificial sequence
  Cleavage product of defensin
YSACPIF

SEQ ID NO: 42 - protein, 6 aa, Artificial sequence
  Cleavage product of defensin
SACPIF

SEQ ID NO: 43 - protein, 5 aa, Artificial sequence
  Cleavage product of defensin
RGKAK

SEQ ID NO: 44 - protein, 5 aa, Artificial sequence
  Cleavage product of defensin
AKCCK

SEQ ID NO: 45 - protein, 36 aa, Homo sapiens; identical to SEQ ID NO: 1

SEQ ID NO: 46 - protein, 33 aa, Artificial sequence; identical to SEQ ID NO: 2
  Cleavage product of defensin

SEQ ID NO: 47 - protein, 33 aa, Artificial sequence; identical to SEQ ID NO: 3
  Cleavage product of defensin

SEQ ID NO: 48 - protein, 31 aa, Artificial sequence; identical to SEQ ID NO: 4
  Cleavage product of defensin

SEQ ID NO: 49 - protein, 29 aa, Artificial sequence
  Cleavage product of defensin
DHYNCVSSGGQCLYSACPIFTKIQGTCYR

SEQ ID NO: 50 - protein, 28 aa, Artificial sequence
  Cleavage product of defensin
DHYNCVSSGGQCLYSACPIFTKIQGTCY

SEQ ID NO: 51 - protein, 28 aa, Artificial sequence
  Cleavage product of defensin
NCVSSGGQCLYSACPIFTKIQGTCYRGK

SEQ ID NO: 52 - protein, 26 aa, Artificial sequence
  Cleavage product of defensin
NCVSSGGQCLYSACPIFTKIQGTCYR

SEQ ID NO: 53 - protein, 25 aa, Artificial sequence
  Cleavage product of defensin
NCVSSGGQCLYSACPIFTKIQGTCY

SEQ ID NO: 54 - protein, 23 aa, Artificial sequence
  Cleavage product of defensin
YSACPIFTKIQGTCYRGKAKCCK

SEQ ID NO: 55 - protein, 22 aa, Artificial sequence
  Cleavage product of defensin
DHYNCVSSGGQCLYSACPIFTK

SEQ ID NO: 56 - protein, 22 aa, Artificial sequence
  Cleavage product of defensin
SACPIFTKIQGTCYRGKAKCCK

SEQ ID NO: 57 - protein, 20 aa, Artificial sequence
  Cleavage product of defensin
DHYNCVSSGGQCLYSACPIF

SEQ ID NO: 58 - protein, 19 aa, Artificial sequence
  Cleavage product of defensin
SACPIFTKIQGTCYRGKAK

SEQ ID NO: 59 - protein, 19 aa, Artificial sequence
  Cleavage product of defensin
NCVSSGGQCLYSACPIFTK

SEQ ID NO: 60 - protein, 17 aa, Artificial sequence
  Cleavage product of defensin
SACPIFTKIQGTCYRGK

SEQ ID NO: 61 - protein, 16 aa, Artificial sequence
  Cleavage product of defensin
TKIQGTCYRGKAKCCK

SEQ ID NO: 62 - protein, 17 aa, Artificial sequence
  Cleavage product of defensin
NCVSSGGQCLYSACPIF

SEQ ID NO: 63 - protein, 16 aa, Artificial sequence
  Cleavage product of defensin
TKIQGTCYRGKAKCCK

SEQ ID NO: 64 - protein, 15 aa, Artificial sequence
  Cleavage product of defensin
SACPIFTKIQGTCYR

SEQ ID NO: 65 - protein, 15 aa, Artificial sequence
  Cleavage product of defensin
YSACPIFTKIQGTCY

SEQ ID NO: 66 - protein, 14 aa, Artificial sequence
  Cleavage product of defensin
IQGTCYRGKAKCCK

SEQ ID NO: 67 - protein, 14 aa, Artificial sequence
  Cleavage product of defensin
DHYNCVSSGGQCLY

SEQ ID NO: 68 - protein, 14 aa, Artificial sequence
  Cleavage product of defensin
IQGTCYRGKAKCCK

SEQ ID NO: 69 - protein, 14 aa, Artificial sequence
  Cleavage product of defensin
SACPIFTKIQGTCY

SEQ ID NO: 70 - protein, 13 aa, Artificial sequence
  Cleavage product of defensin
DHYNCVSSGGQCL

SEQ ID NO: 71 - protein, 11 aa, Artificial sequence
  Cleavage product of defensin
NCVSSGGQCLY

SEQ ID NO: 72 - protein, 13 aa, Artificial sequence
  Cleavage product of defensin
TKIQGTCYRGKAK

SEQ ID NO: 73 - protein, 11 aa, Artificial sequence
  Cleavage product of defensin
TKIQGTCYRGK

SEQ ID NO: 74 - protein, 11 aa, Artificial sequence
  Cleavage product of defensin
IQGTCYRGKAK

SEQ ID NO: 75 - protein, 11 aa, Artificial sequence
  Cleavage product of defensin
NCVSSGGQCLY

SEQ ID NO: 76 - protein, 10 aa, Artificial sequence
  Cleavage product of defensin
NCVSSGGQCL

SEQ ID NO: 77 - protein, 9 aa, Artificial sequence
  Cleavage product of defensin
TKIQGTCYR

SEQ ID NO: 78 - protein, 9 aa, Artificial sequence
  Cleavage product of defensin
IQGTCYRGK

SEQ ID NO: 79 - protein, 8 aa, Artificial sequence
  Cleavage product of defensin
TKIQGTCY

SEQ ID NO: 80 - protein, 8 aa, Artificial sequence
  Cleavage product of defensin
RGKAKCCK

SEQ ID NO: 81 - protein, 8 aa, Artificial sequence
  Cleavage product of defensin
SACPIFTK

SEQ ID NO: 82 - protein, 7 aa, Artificial sequence
  Cleavage product of defensin
IQGTCYR

SEQ ID NO: 83 - protein, 7 aa, Artificial sequence
  Cleavage product of defensin
GKAKCCK

SEQ ID NO: 84 - protein, 6 aa, Artificial sequence
  Cleavage product of defensin
IQGTCY

SEQ ID NO: 85 - protein, 7 aa, Artificial sequence
  Cleavage product of defensin
YSACPIF

SEQ ID NO: 86 - protein, 6 aa, Artificial sequence
  Cleavage product of defensin
SACPIF

SEQ ID NO: 87 - protein, 5 aa, Artificial sequence
  Cleavage product of defensin
RGKAK

SEQ ID NO: 88 - protein, 5 aa, Artificial sequence
  Cleavage product of defensin
AKCCK

SEQ ID NO: 89 - protein, 32 aa, Homo sapiens
ATCYCRTGRCATRESLSGVCEISGRLYRLCCR

SEQ ID NO: 90 - protein, 29 aa, Artificial sequence
  Cleavage product of defensin
ATCYCRTGRCATRESLSGVCEISGRLYRL

SEQ ID NO: 91 - protein, 28 aa, Artificial sequence
  Cleavage product of defensin
CRTGRCATRESLSGVCEISGRLYRLCCR

SEQ ID NO: 92 - protein, 28 aa, Artificial sequence
  Cleavage product of defensin
ATCYCRTGRCATRESLSGVCEISGRLYR

SEQ ID NO: 93 - protein, 27 aa, Artificial sequence
  Cleavage product of defensin
ATCYCRTGRCATRESLSGVCEISGRLY

SEQ ID NO: 94 - protein, 26 aa, Artificial sequence
  Cleavage product of defensin
TGRCATRESLSGVCEISGRLYRLCCR

SEQ ID NO: 95 - protein, 26 aa, Artificial sequence
  Cleavage product of defensin
ATCYCRTGRCATRESLSGVCEISGRL

SEQ ID NO: 96 - protein, 25 aa, Artificial sequence
  Cleavage product of defensin
CRTGRCATRESLSGVCEISGRLYRL

SEQ ID NO: 97 - protein, 25 aa, Artificial sequence
  Cleavage product of defensin
ATCYCRTGRCATRESLSGVCEISGR

SEQ ID NO: 98 - protein, 24 aa, Artificial sequence
  Cleavage product of defensin
CRTGRCATRESLSGVCEISGRLYR

SEQ ID NO: 99 - protein, 23 aa, Artificial sequence
  Cleavage product of defensin
CATRESLSGVCEISGRLYRLCCR

SEQ ID NO: 100 - protein, 23 aa, Artificial sequence
  Cleavage product of defensin
CRTGRCATRESLSGVCEISGRLY

SEQ ID NO: 101 - protein, 22 aa, Artificial sequence
  Cleavage product of defensin
TGRCATRESLSGVCEISGRLYR

SEQ ID NO: 102 - protein, 22 aa, Artificial sequence
  Cleavage product of defensin
CRTGRCATRESLSGVCEISGRL

SEQ ID NO: 103 - protein, 21 aa, Artificial sequence
  Cleavage product of defensin
TGRCATRESLSGVCEISGRLY

SEQ ID NO: 104 - protein, 21 aa, Artificial sequence
  Cleavage product of defensin
CRTGRCATRESLSGVCEISGR

SEQ ID NO: 105 - protein, 19 aa, Artificial sequence
  Cleavage product of defensin
ESLSGVCEISGRLYRLCCR

SEQ ID NO: 106 - protein, 19 aa, Artificial sequence
  Cleavage product of defensin
CATRESLSGVCEISGRLYR

SEQ ID NO: 107 - protein, 19 aa, Artificial sequence
  Cleavage product of defensin
TGRCATRESLSGVCEISGR

SEQ ID NO: 108 - protein, 18 aa, Artificial sequence
  Cleavage product of defensin
CATRESLSGVCEISGRLY

SEQ ID NO: 109 - protein, 16 aa, Artificial sequence
  Cleavage product of defensin
SGVCEISGRLYRLCCR

SEQ ID NO: 110 - protein, 16 aa, Artificial sequence
  Cleavage product of defensin
ATCYCRTGRCATRESL

SEQ ID NO: 111 - protein, 15 aa, Artificial sequence
  Cleavage product of defensin
ESLSGVCEISGRLYR

SEQ ID NO: 112 - protein, 16 aa, Artificial sequence
  Cleavage product of defensin
CATRESLSGVCEISGR

SEQ ID NO: 113 - protein, 14 aa, Artificial sequence
  Cleavage product of defensin
ESLSGVCEISGRLY

SEQ ID NO: 114 - protein, 13 aa, Artificial sequence
  Cleavage product of defensin
ATCYCRTGRCATR

SEQ ID NO: 115 - protein, 13 aa, Artificial sequence
  Cleavage product of defensin
SGVCEISGRLYRL

SEQ ID NO: 116 - protein, 12 aa, Artificial sequence
  Cleavage product of defensin
CRTGRCATRESL

SEQ ID NO: 117 - protein, 12 aa, Artificial sequence
  Cleavage product of defensin
ESLSGVCEISGR

SEQ ID NO: 118 - protein, 11 aa, Artificial sequence
  Cleavage product of defensin
SGVCEISGRLY

SEQ ID NO: 119 - protein, 9 aa, Artificial sequence
  Cleavage product of defensin
ATCYCRTGR

SEQ ID NO: 120 - protein, 9 aa, Artificial sequence
  Cleavage product of defensin
CRTGRCATR

SEQ ID NO: 121 - protein, 10 aa, Artificial sequence
  Cleavage product of defensin
SGVCEISGRL

SEQ ID NO: 122 - protein, 7 aa, Artificial sequence
  Cleavage product of defensin
LYRLCCR

SEQ ID NO: 123 - protein, 6 aa, Artificial sequence
  Cleavage product of defensin
YRLCCR

SEQ ID NO: 124 - protein, 7 aa, Artificial sequence
  Cleavage product of defensin
TGRCATR

SEQ ID NO: 125 - protein, 6 aa, Artificial sequence
  Cleavage product of defensin
ATCYCR

SEQ ID NO: 126 - protein, 5 aa, Artificial sequence
  Cleavage product of defensin
RLCCR

SEQ ID NO: 127 - protein, 5 aa, Artificial sequence
  Cleavage product of defensin
CRTGR

SEQ ID NO: 128 - protein, 32 aa, Homo sapiens
AFTCHCRRSCYSTEYSYGTCTVMGINHRFCCL

SEQ ID NO: 129 - protein, 30 aa, Artificial sequence
  Cleavage product of defensin
TCHCRRSCYSTEYSYGTCTVMGINHRFCCL

SEQ ID NO: 130 - protein, 29 aa, Artificial sequence
  Cleavage product of defensin
AFTCHCRRSCYSTEYSYGTCTVMGINHRF

SEQ ID NO: 131 - protein, 28 aa, Artificial sequence
  Cleavage product of defensin
AFTCHCRRSCYSTEYSYGTCTVMGINHR

SEQ ID NO: 132 - protein, 27 aa, Artificial sequence
  Cleavage product of defensin
TCHCRRSCYSTEYSYGTCTVMGINHRF

SEQ ID NO: 133 - protein, 26 aa, Artificial sequence
  Cleavage product of defensin
TCHCRRSCYSTEYSYGTCTVMGINHR

SEQ ID NO: 134 - protein, 25 aa, Artificial sequence
  Cleavage product of defensin
RSCYSTEYSYGTCTVMGINHRFCCL

SEQ ID NO: 135 - protein, 24 aa, Artificial sequence
  Cleavage product of defensin
SCYSTEYSYGTCTVMGINHRFCCL

SEQ ID NO: 136 - protein, 23 aa, Artificial sequence
  Cleavage product of defensin
AFTCHCRRSCYSTEYSYGTCTVM

SEQ ID NO: 137 - protein, 22 aa, Artificial sequence
  Cleavage product of defensin
RSCYSTEYSYGTCTVMGINHRF

SEQ ID NO: 138 - protein, 21 aa, Artificial sequence
  Cleavage product of defensin
TCHCRRSCYSTEYSYGTCTVM

SEQ ID NO: 139 - protein, 21 aa, Artificial sequence
  Cleavage product of defensin
RSCYSTEYSYGTCTVMGINHR

SEQ ID NO: 140 - protein, 21 aa, Artificial sequence
  Cleavage product of defensin
SCYSTEYSYGTCTVMGINHRF

SEQ ID NO: 141 - protein, 21 aa, Artificial sequence
  Cleavage product of defensin
STEYSYGTCTVMGINHRFCCL

SEQ ID NO: 142 - protein, 20 aa, Artificial sequence
  Cleavage product of defensin
SCYSTEYSYGTCTVMGINHR

SEQ ID NO: 143 - protein, 17 aa, Artificial sequence
  Cleavage product of defensin
AFTCHCRRSCYSTEYSY

SEQ ID NO: 144 - protein, 18 aa, Artificial sequence
  Cleavage product of defensin
STEYSYGTCTVMGINHRF

SEQ ID NO: 145 - protein, 17 aa, Artificial sequence
  Cleavage product of defensin
STEYSYGTCTVMGINHR

SEQ ID NO: 146 - protein, 17 aa, Artificial sequence
  Cleavage product of defensin
SYGTCTVMGINHRFCCL

SEQ ID NO: 147 - protein, 15 aa, Artificial sequence
  Cleavage product of defensin
TCHCRRSCYSTEYSY

SEQ ID NO: 148 - protein, 15 aa, Artificial sequence
  Cleavage product of defensin
AFTCHCRRSCYSTEY

SEQ ID NO: 149 - protein, 15 aa, Artificial sequence
  Cleavage product of defensin
GTCTVMGINHRFCCL

SEQ ID NO: 150 - protein, 13 aa, Artificial sequence
  Cleavage product of defensin
TCHCRRSCYSTEY

SEQ ID NO: 151 - protein, 14 aa, Artificial sequence
  Cleavage product of defensin
SYGTCTVMGINHRF

SEQ ID NO: 152 - protein, 13 aa, Artificial sequence
  Cleavage product of defensin
SYGTCTVMGINHR

SEQ ID NO: 153 - protein, 11 aa, Artificial sequence
  Cleavage product of defensin
AFTCHCRRSCY

SEQ ID NO: 154 - protein, 12 aa, Artificial sequence
  Cleavage product of defensin
STEYSYGTCTVM

SEQ ID NO: 155 - protein, 12 aa, Artificial sequence
  Cleavage product of defensin
GTCTVMGINHRF

SEQ ID NO: 156 - protein, 10 aa, Artificial sequence
  Cleavage product of defensin
RSCYSTEYSY

SEQ ID NO: 157 - protein, 11 aa, Artificial sequence
  Cleavage product of defensin
GTCTVMGINHR

SEQ ID NO: 158 - protein, 9 aa, Artificial sequence
  Cleavage product of defensin
TCHCRRSCY

SEQ ID NO: 159 - protein, 9 aa, Artificial sequence
  Cleavage product of defensin
SCYSTEYSY

SEQ ID NO: 160 - protein, 9 aa, Artificial sequence
  Cleavage product of defensin
GINHRFCCL

SEQ ID NO: 161 - protein, 8 aa, Artificial sequence
  Cleavage product of defensin
RSCYSTEY

SEQ ID NO: 162 - protein, 8 aa, Artificial sequence
  Cleavage product of defensin
AFTCHCRR

SEQ ID NO: 163 - protein, 8 aa, Artificial sequence
  Cleavage product of defensin
SYGTCTVM

SEQ ID NO: 164 - protein, 7 aa, Artificial sequence
  Cleavage product of defensin
SCYSTEY

SEQ ID NO: 165 - protein, 7 aa, Artificial sequence
  Cleavage product of defensin
AFTCHCR

SEQ ID NO: 166 - protein, 6 aa, Artificial sequence
  Cleavage product of defensin
TCHCRR

SEQ ID NO: 167 - protein, 6 aa, Artificial sequence
  Cleavage product of defensin
STEYSY

SEQ ID NO: 168 - protein, 6 aa, Artificial sequence
  Cleavage product of defensin
GINHRF

SEQ ID NO: 169 - protein, 5 aa, Artificial sequence
  Cleavage product of defensin
TCHCR

SEQ ID NO: 170 - protein, 6 aa, Artificial sequence
  Cleavage product of defensin
GTCTVM

SEQ ID NO: 171 - protein, 4 aa, Artificial sequence
  Cleavage product of defensin
RSCY

SEQ ID NO: 172 - protein, 33 aa, Homo sapiens
VCSCRLVFCRRTELRVGNCLIGGVSFTYCCTRV

SEQ ID NO: 173 - protein, 32 aa, Artificial sequence
  Cleavage product of defensin
VCSCRLVFCRRTELRVGNCLIGGVSFTYCCTR

SEQ ID NO: 174 - protein, 28 aa, Artificial sequence
  Cleavage product of defensin
VCSCRLVFCRRTELRVGNCLIGGVSFTY

SEQ ID NO: 175 - protein, 28 aa, Artificial sequence
  Cleavage product of defensin
LVFCRRTELRVGNCLIGGVSFTYCCTRV

SEQ ID NO: 176 - protein, 27 aa, Artificial sequence
  Cleavage product of defensin
LVFCRRTELRVGNCLIGGVSFTYCCTR

SEQ ID NO: 177 - protein, 26 aa, Artificial sequence
  Cleavage product of defensin
VCSCRLVFCRRTELRVGNCLIGGVSF

SEQ ID NO: 178 - protein, 25 aa, Artificial sequence
  Cleavage product of defensin
CRRTELRVGNCLIGGVSFTYCCTRV

SEQ ID NO: 179 - protein, 24 aa, Artificial sequence
  Cleavage product of defensin
CRRTELRVGNCLIGGVSFTYCCTR

SEQ ID NO: 180 - protein, 23 aa, Artificial sequence
  Cleavage product of defensin
LVFCRRTELRVGNCLIGGVSFTY

SEQ ID NO: 181 - protein, 23 aa, Artificial sequence
  Cleavage product of defensin
RTELRVGNCLIGGVSFTYCCTRV

SEQ ID NO: 182 - protein, 22 aa, Artificial sequence
  Cleavage product of defensin
RTELRVGNCLIGGVSFTYCCTR

SEQ ID NO: 183 - protein, 22 aa, Artificial sequence
  Cleavage product of defensin
TELRVGNCLIGGVSFTYCCTRV

SEQ ID NO: 184 - protein, 21 aa, Artificial sequence
  Cleavage product of defensin
LVFCRRTELRVGNCLIGGVSF

SEQ ID NO: 185 - protein, 21 aa, Artificial sequence
  Cleavage product of defensin
TELRVGNCLIGGVSFTYCCTR

SEQ ID NO: 186 - protein, 20 aa, Artificial sequence
  Cleavage product of defensin
CRRTELRVGNCLIGGVSFTY

SEQ ID NO: 187 - protein, 18 aa, Artificial sequence
  Cleavage product of defensin
RTELRVGNCLIGGVSFTY

SEQ ID NO: 188 - protein, 18 aa, Artificial sequence
  Cleavage product of defensin
CRRTELRVGNCLIGGVSF

SEQ ID NO: 189 - protein, 18 aa, Artificial sequence
  Cleavage product of defensin
VGNCLIGGVSFTYCCTRV

SEQ ID NO: 190 - protein, 15 aa, Artificial sequence
  Cleavage product of defensin
VCSCRLVFCRRTELR

SEQ ID NO: 191 - protein, 17 aa, Artificial sequence
  Cleavage product of defensin
TELRVGNCLIGGVSFTY

SEQ ID NO: 192 - protein, 17 aa, Artificial sequence
  Cleavage product of defensin
VGNCLIGGVSFTYCCTR

SEQ ID NO: 193 - protein, 16 aa, Artificial sequence
  Cleavage product of defensin
RTELRVGNCLIGGVSF

SEQ ID NO: 194 - protein, 15 aa, Artificial sequence
  Cleavage product of defensin
TELRVGNCLIGGVSF

SEQ ID NO: 195 - protein, 11 aa, Artificial sequence
  Cleavage product of defensin
VCSCRLVFCRR

SEQ ID NO: 196 - protein, 13 aa, Artificial sequence
  Cleavage product of defensin
VGNCLIGGVSFTY

SEQ ID NO: 197 - protein, 10 aa, Artificial sequence
  Cleavage product of defensin
LVFCRRTELR

SEQ ID NO: 198 - protein, 10 aa, Artificial sequence
  Cleavage product of defensin
VCSCRLVFCR

SEQ ID NO: 199 - protein, 11 aa, Artificial sequence
  Cleavage product of defensin
VGNCLIGGVSF

SEQ ID NO: 200 - protein, 7 aa, Artificial sequence
  Cleavage product of defensin
CRRTELR

SEQ ID NO: 201 - protein, 8 aa, Artificial sequence
  Cleavage product of defensin
VCSCRLVF

SEQ ID NO: 202 - protein, 7 aa, Artificial sequence
  Cleavage product of defensin
TYCCTRV

SEQ ID NO: 203 - protein, 6 aa, Artificial sequence
  Cleavage product of defensin
LVFCRR

SEQ ID NO: 204 - protein, 6 aa, Artificial sequence
  Cleavage product of defensin
TYCCTR

SEQ ID NO: 205 - protein, 5 aa, Artificial sequence
  Cleavage product of defensin
RTELR

SEQ ID NO: 206 - protein, 5 aa, Artificial sequence
  Cleavage product of defensin
LVFCR

SEQ ID NO: 207 - protein, 5 aa, Artificial sequence
  Cleavage product of defensin
CCTRV

SEQ ID NO: 208 - protein, 5 aa, Artificial sequence
  Cleavage product of defensin
VCSCR

SEQ ID NO: 209 - protein, 4 aa, Artificial sequence
  Cleavage product of defensin
TELR

(19) United States
(12) Patent Application Publication
(10) Pub. No.: US 2023/0365639 A1
(43) Pub. Date: Nov. 16, 2023

(54) DEFENSIN FRAGMENT DERIVED LIPOPEPTIDES FOR TREATMENT OF DRUG-RESISTANT MICROORGANISMS

(71) Applicant: AESCULUS BIO APS, Copenhagen N (DK)

(72) Inventors: Jan Wehkamp, Hopewell, NJ (US); Louis Köninger, Tübingen (DE)

(21) Appl. No.: 18/044,802

(22) PCT Filed: Sep. 14, 2021
(86) PCT No.: PCT/EP2021/075198

§ 371 Date: Mar. 10, 2023

(30) Foreign Application Priority Data
Sep. 14, 2020 (EP) 20195919.4

(51) Int. Cl. C07K 14/47; A61K 47/54; A61P 31/00; A61K 47/65; A61K 47/10; A61K 47/26

(52) U.S. Cl. CPC C07K 14/4723; A61K 47/542; A61P 31/00; A61K 47/65; A61K 47/10; A61K 47/26

(57) ABSTRACT

The present invention relates to development of innovative antibiotics against drug resistant bacteria, viruses, protozoa, fungi or worms based on coupling of lipopeptides to fragments of human defensins to achieve high antimicrobial efficacy and biofilm degradation as well as low development of resistance while preserving the natural microbiota thus not only eradicating bacterial, viral, protozoal, fungal or worm infection but preventing a number of disorders associated with antibiotic treatment e.g. infection with C. difficile'.

### TECHNICAL FIELD

The present invention relates to development of antibiotics against drug resistant pathogens based on coupling of fatty acids to fragments of defensins to achieve high antimicrobial efficacy while preserving the natural microbiota.

### BACKGROUND

Antibiotic-resistant bacteria are an urgent and growing public health threat. Specifically, the so-called ESKAPE (Enterococcus faecium, Staphylococcus aureus, Klebsiella pneumoniae, Acinetobacter baumannii, Pseudomonas aeruginosa, and Enterobacter species) pathogens account for the majority of nosocomial infections worldwide being attributed to ≥700,000 deaths annually (Kelly and Davies, 2017). The World Health Organization (WHO) has recently published a list of 12 bacteria against which new antibiotics are urgently needed, including the ESKAPE pathogens (Tacconelli et al., 2018). While traditional antibiotics fight pathogens, they also have wide-ranging consequences for the commensal gut microbiota (Maier et al., 2020). Administration disrupts the microbial composition and can result in a long-lasting dysbiosis, which is associated with an increasing number of diseases (Jackson et al., 2018). Decreased diversity and -taxonomic richness, the spread of antimicrobial resistance as well as increased colonization of opportunistic pathogens, including secondary infections with Clostridium difficile, are just a few of the many side-effects traditional antibiotics impose (Francino, 2016; Kim et al., 2017). The current antimicrobial crisis is a product of the long-term neglected development of new antibiotics by pharmaceutical companies and governments (The Lancet, 2020). Thus, new strategies more resilient to multidrug resistance but importantly preserving the natural microbiota are urgently warranted (Falagas et al., 2016).

Antimicrobial peptides (AMPs) are small, cationic peptides existing in all multicellular organisms exhibiting a broad range of antimicrobial and immunological properties (Zasloff, 2002). Defensins, the most prominent class of AMPs in humans, protect the host against infectious microbes and shape the composition of microbiota at mucosal surfaces (Bevins, 2003; Ganz, 2003; Peschel and Sahl, 2006; Thaiss et al., 2016). Defensins have been classified in three groups α, β and θ defensins. The β-defensins and the α-defensins HD5 and HD6 are expressed in surface epithelia by monocytes, plasmacytoid dendritic cells and platelets. The α-defensins HNP1-4 are expressed in leucocytes. Most of the defensins cannot be degraded but the α-defensins HD5, HNP-4 and the β-defensin hBD-1 can be degraded into a number of biologically active fragments by intestinal proteases. Previously, the antimicrobial activity of hBD-1, which is constitutively expressed on all bodily surfaces, was underestimated until it was analyzed under reduced conditions as found in the human intestine. Reduced hBD-1 surprisingly exhibited increased antimicrobial activity, but could be degraded by intestinal proteases (Raschig et al., 2017; Schroeder et al., 2011). It has recently been demonstrated that the reduction of hBD-1 among others creates an eight-amino acid carboxyl-terminal fragment (octapeptide) with retained antimicrobial activity but with low in vivo stability (Wendler et al., 2019).

### SUMMARY

The inventors have developed a group of novel and stabile synthetic lipopeptides with improved antimicrobial activity and preservation of the natural microbiota. The inventors have modified an hBD-1-derived octapeptide with lipids e.g. palmitic acid and various spacers, such as sugars or amino acids, to create lipopeptides (Pams) with increased stability and bactericidal activity while preserving the gastrointestinal microbiota following oral administration.

The inventors have demonstrated that the Pams are highly efficacious against ESKAPE pathogens as well as against C. albicans and C. tropicalis. It has further been demonstrated that the Pams are efficacious at eradicating bacterially produced biofilms and that resistance development is surprisingly negligible. The inventors have demonstrated that the mode of action is cell envelope damage with disruption of the cell membrane and pore formation as well as breakdown of the cell membrane potential.

Strong antimicrobial effect is commonly associated with toxicity. The inventors have demonstrated that despite strong antimicrobial efficacy the Pams are associated with no or little cytotoxicity. Safety was tested in vivo by oral administration of Pam-3. No danger signals neither chemical nor histologically were identified from kidney, liver or the gastrointestinal tract.

In vivo efficacy was tested in both an acute gastrointestinal infection caused by S. Typhimurium and an established gastrointestinal infection caused by C. rodentium comparing the least efficacious Pam-1 with the most efficacious Pam-3. Pam-3 showed highly significant reduction of CFU's in both models and both in gut content and in gut tissue whereas Pam-1 only showed significant CFU reduction in the acute model and this only for gut content.

The inventors have surprisingly demonstrated that despite the high antimicrobial efficacy against pathogenic bacteria, no changes in the microbiota composition before and after oral treatment even with the most efficacious Pam-3 was observed. Thus, the number of species and complexity remained comparable to PBS-treated controls and the treatment did not affect the abundance of bacterial genera.

### DESCRIPTION OF DRAWINGS

FIG. 1 shows the chemical structure of the Octapeptide, the C-terminal eight amino acids of human beta-defensin 1, chemically modified with palmitic acid and different spacers such as sugars or amino acids or 8-amino-3.6-dioxaoctanoic acid (Ado) to generate lipopeptides:
Pam1: Pam2-Glc-Suc-RGKAKCCK
Pam2: Pam-RGKAKCCK
Pam3: Pam-Ado-RGKAKCCK
Pam4: Pam3Cys-RGKAKCCK
Pam5: Pam-Lys(Pam)-RGKAKCCK

FIG. 2 shows the antimicrobial activity of all five Pams as well as the octapeptide arising from reduction of hBD-1 against S. aureus; C. rodentium; P. aeruginosa; S. Typhimurium; E. faecalis; E. faecium; E. coli and K. pneumoniae as determined by radial diffusion assay. 1 μg of each peptide was used. The diameter of inhibition zones indicates antimicrobial activity; a diameter of 2.5 mm (dotted line) is the diameter of an empty well. Results are medians of three independent experiments.

FIG. 3 shows the Minimal Inhibitory Concentration (MIC) of all five Pams against three different strains of S. aureus; A. baumannii; P. aeruginosa; E. faecium; E. coli and K. pneumoniae. Broth microdilution assay with the Pam's against bacteria from the ESKAPE panel. FIG. 3 shows the detailed results of the broth microdilution assay experiments. The dotted line marks the highest peptide concentration used in these experiments. Data are presented as mean±SEM. Experiments were carried out three independent times.

FIG. 4 shows the antifungal activity of all five Pams against C. albicans and C. tropicalis as determined by broth microdilution assay. Data are presented as mean±SEM. Experiments were carried out three independent times.

FIG. 5 shows eradication of biofilm produced by P. aeruginosa by Pam-3. Bactericidal activity of Pam-3 against established biofilms of S. aureus ATCC25923 (black line) and P. aeruginosa PAO1 (grey line). Results are expressed as the number of viable bacteria (in log₁₀ CFU) after 1 hour treatment of 24 hours old biofilms with Pam-3. Values are means of three replicates from three independent experiments. Statistics were evaluated by using the Kruskal-Wallis test comparing each timepoint against timepoint 0 (*p<0.05, **p<0.01, ***p<0.001, and ****p<0.0001).

FIG. 6 corroborates lack of development of bacterial resistance against Pam-3 as assessed against S. aureus and S. Typhimurium. Resistance development of S. aureus ATCC 25923 and S. Typhimurium DSM554 to Pam-3 and the antibiotics rifampicin and ciprofloxacin, respectively. Values are fold changes (in log₂) in minimal inhibitory concentration (MIC) relative to the MIC of the first passage.

FIG. 7 shows cell envelope damage after exposure to the octapeptide and all five Pams ascertained by reporter gene assay.

FIG. 8 shows reduction of bacterial membrane potential by the octapeptide and all five Pams compared with the protonophore carbonyl cyanide m-chlorophenyl hydrazone CCCP as certained by membrane potential assay.

FIG. 9 shows bacterial pore formation as determined by coloring with Syto9 and propidium iodide respectively.

FIG. 10 shows the rapidness of Pam-3 mediated killing. Pam-3 killed more than 90% of P. aeruginosa within 1 min and S. aureus within 15 min. Killing of S. aureus ATCC25923 (black line) and P. aeruginosa PAO1 (grey line) after 1 to 120 min exposure to 9.38 μM (1× MIC) Pam-3. Results are expressed as the number of viable bacteria (in log₁₀ CFU) per milliliter. Values are means of three independent experiments.

FIG. 11 shows cell envelope damage and pore formation of S. aureus caused by Pam-3 after 30 or 60 min incubation at 4 times MIC. Scale bars of all TEM pictures are 0.2 μm and 2 μm of all SEM pictures.

FIG. 12 shows cell envelope damage and pore formation of S. aureus caused by the five different octapeptide derived lipopeptides after 120 min as determined by Transmission and Scanning Electron Microscopy. Here, an incubation time of 120 min was used to be in line with the incubation time of the broth microdilution assay. Scale bars of all TEM pictures are 0.5 μm and 2 μm of all SEM pictures.

FIG. 13 shows cell envelope damage and pore formation of S. Enteritidis caused by the five octapeptide derived lipopeptides after 120 min as determined by Scanning Electron Microscopy. Here, an incubation time of 120 min was used to be in line with the incubation time of the broth microdilution assay. Scale bars of all TEM pictures are 0.5 μm and 2 μm of all SEM pictures.

FIG. 14 shows MIC of Pam-1 to Pam-5 against E. coli LPS mutants.

FIG. 15 shows MIC of Pam-1 to Pam-5 against S. aureus cell envelope mutants.

FIG. 16 shows cytotoxicity as determined by LHD assay.

FIG. 17 shows cytotoxicity as determined by WST-1 cell proliferation assay.

FIG. 18 shows cytotoxicity as determined by hemolysis of red blood cell test.

FIG. 19 shows bodyweight, glutamic oxaloacetic transaminase and creatinine levels after 125 and 250 μg of Pam-1 and Pam-3 respectively. Dose dependent oral tolerance test in mice. Animals were treated twice with 125 μg or once with 250 μg of Pam-1, Pam-3 or PBS. (a) Weight change of mice, (b) and glutamic oxaloacetic transaminase and (c) creatinine levels one day after Pam-1 or Pam-3 application. Results are presented as mean±SEM. Oral tolerance test in mice. Animals were treated twice with 250 μg Pam-1, Pam-3 or PBS. Representative images from the stomach, small intestine (jejunum), cecum and colon are shown. Scale bars, 50 μm.

FIG. 20 shows bacterial load (CFU's) in gut content/gut tissue of cecum and small intestine following treatment of acute gastrointestinal infection with S. Typhimurium and established gastrointestinal infection with C. rodentium, respectively, with Pam-1. Mice were infected with S. Typhimurium and treated orally with either 250 μg Pam-1 (N=11) or PBS (N=9) after 6 hours and 22 hours post infection. Mice were infected with C. rodentium and treated orally with either 250 μg Pam-1 (N=10 or 11) or PBS (N=11) after 5 days post infection.

FIG. 21 shows body weight, bacterial load (CFU's) in gut content/tissue of cecum and small intestine following treatment of acute gastrointestinal infection with S. Typhimurium and established gastrointestinal infection with C. rodentium, respectively, with Pam-3. Mice were infected with S. Typhimurium and treated orally with either 250 μg Pam-3 (N=12) or PBS (N=9) after 6 hours and 22 hours post infection. Mice were infected with C. rodentium and treated orally with either 250 μg Pam-3 (N=11 or 14) or PBS (N=11) after 5 days post infection. (a) Body weight change during acute S. Typhimurium infection. (b) CFU/ml of S. Typhimurium in cecum content and tissue. (c) CFU/ml of S. Typhimurium in small intestine content and tissue. (d) CFU/g of C. rodentium in cecum content and tissue. (e) CFU/g of C. rodentium in colon content and tissue. Results are expressed as the number of viable bacteria (in logo CFU) in the lumen and tissue and presented as mean±SEM, significantly different as compared to the PBS group, as calculated using the Mann-Whitney test.

FIG. 22 is a detailed microbiome analysis including weighted and unweighted unifrac analysis, observed species and relative abundance of bacteria before and after treatment with Pam-1 respectively. Chow-fed mice were treated orally twice with 125 μg Pam-1 (N=5) or once with 250 μg Pam-1 (N=5-6) or PBS (N=5) as a control. Feces samples were collected before and after treatment to observe short term changes in the microbiome. (a) Principal coordinate analysis (PCoA including group mean) of fecal microbiota composition using Weighted UniFrac Distances before and after treatment, respectively. (b) PCoA including group mean of fecal microbiota composition using Unweighted UniFrac Distances, respectively before and after treatment. (c) Richness (observed species) before and after treatment. (d) Fecal microbiota was calculated by Shannon's Diversity index. The statistical significance was calculated by using Wilcoxon test. (e) Pam-1 treatment affects the abundance of bacterial genera.

[symbol] g is a detailed microbiome analysis including weighted and unweighted unifrac analysis, observed species and relative abundance of bacteria before and after treatment with Pam-3 respectively. Chow-fed mice were treated orally twice with 125 μg Pam-3 (N=5) or 250 μg Pam-3 (N=6) or PBS (N=5) as a control. Feces samples were collected before and after treatment to observe short term changes in the microbiome. (a) Principal coordinate analysis (PCoA including group mean) of fecal microbiota composition using Weighted UniFrac Distances before and after treatment, respectively. (b) PCoA including group mean of fecal microbiota composition using Unweighted UniFrac Distances, respectively before and after treatment. (c) Richness (observed species) before and after treatment. (d) Fecal microbiota was calculated by Shannon's Diversity index. The statistical significance was calculated by using Wilcoxon test. (e) Pam-3 treatment affects the abundance of bacterial genera. (f) Aggregated by genus. Statistical analysis performed by the LEfSe platform ((https://galaxyproject.org/learn/visualization/custom/lefse/) using default settings.

FIG. 24 shows a microbiome analysis after treatment with ampicillin.

FIG. 25 shows the bactericidal effect of all the five Pams as determined by broth microdilution assay.

FIG. 26 shows histological scoring liver, kidney and gastrointestinal tract of mice treated with Pam-1, Pam-3 and PBS respectively.

FIG. 27. Clustal W (2.1) multiple sequence alignment of human beta defensin 1-4.

FIG. 28. Clustal W (2.1) multiple sequence alignment of human alpha defensin 5 and 6.

FIG. 29: Clustal W (2.1) multiple sequence alignment of human neutrophil peptide 1-3.

In the Clustal W alignments:
* indicates positions which have a single, fully conserved residue.
: indicates that one of the following ‘strong’ groups is fully conserved: -S,T,A; N,E,Q,K; N,H,Q,K; N,D,E,Q; Q,H,R,K; M,I,L,V; M,I,L,F; H,Y; F,Y,W.
. indicates that one of the following ‘weaker’ groups is fully conserved: -C,S,A; A,T,V; S,A,G; S,T,N,K; S,T,P,A; S,G,N,D; S,N,D,E,Q,K; N,D,E,Q,H,K; N,E,Q,H,R, K; V,L,I,M; H,F,Y.

### DETAILED DESCRIPTION

### Definitions

Defensin fragments: The term “defensin fragment” as used herein refers to peptides belonging to the defensin class of antimicrobial peptides. Most defensins cannot be degraded by gastrointestinal enzymes but the two α-defensins HD5 and HNP4 as well as the β-defensins hBD-1 can be degraded into short linear peptides when subjected to gastrointestinal or bacterial enzymes.

Pams: The term Pam as used herein refers to compounds generated by coupling of a defensin fragment with a fatty acid with or without a spacer such as a sugar of amino acid to generate a compound with antimicrobial properties.

Identity: The relatedness between two amino acid sequences or between two nucleotide sequences is described by the parameter “identity”. The degree of identity between two amino acid sequences is determined using the Needleman-Wunsch algorithm (Needleman and Wunsch, 1970) as implemented in the Needle program of the EMBOSS package (Rice et al., 2000), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows: (Identical Residues×100)/(Length of Alignment−Total Number of Gaps in Alignment).

Normal microbiota: The term “normal microbiota” is used herein to indicate a microbiota that is not dysbiotic. Normal microbiota is characterized by having adequate gene richness.

Normal intestinal microbiota is characterized by comprising bacteria belonging to the genera Bacteriodetes, Firmicutes, Faecalibacterium, Roseburia, Blautia, Ruminococcus, Coprococcus, Bifidobacterium, Methanobrevibacter, Lactobacillus, Coprococcus, Clostridium, Akkermansia, Eubacterium.

Treatment: The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering, reducing the active compounds to prevent or reduce the risk of the onset of symptoms or complications. The patient to be treated is preferably a mammalian, in particular a human being.

Modification: The term “modification” means herein any chemical modification of a mammalian (e.g. human) defensin. The modification(s) can be substitution(s), deletion(s) and/or insertions(s) of the amino acid(s) as well as replacement(s) of amino acid side chain(s); or use of unnatural amino acids with similar characteristics in the amino acid sequence or lipidation(s) as well as the insertion of optional linker/spacer. In particular the modification(s) can be amidations, such as amidation of the C-terminus. Preferably, amino acid modifications are of a minor nature, that is conservative amino acid substitutions or insertions that do not significantly affect the folding and/or activity of the polypeptide; single deletions; small amino- or carboxyl-terminal extensions; or a small extension that facilitates purification by changing net charge or another function, such as a poly-histidine tag, an antigenic epitope or a binding domain. In one embodiment the small extension, such as a poly-histidine tag, an antigenic epitope or a binding domain is attached to the mammalian (e.g. human) alpha or beta defensin through a small linker peptide of up to about 20-25 residues and said linker may contain a restriction enzyme cleavage site.

The Clustal W alignments can be used to predict which amino acid residues can be substituted without substantially affecting the biological activity of the protein. The sequences were aligned using Clustal W2.1 (http://www.genome.jp/tools/clustalw/) and the following settings: Gap Open Penalty:10, Gap Extension Penalty: 0.05, Weight Transition: NO, Hydrophilic Residues for Proteins: GPSNDQE, Hydrophilic Gaps: YES, Weight Matrix: BLOSUM (for PROTEIN). Substitutions within the following group (Clustal W, ‘strong’ conservation group) are to be regarded as conservative substitutions:-S,T,A; N,E,Q,K; N,H,Q,K; N,D,E,Q; Q,H,R,K; M,I,L,V; M,I,L,F; H,Y; F,Y,W. Substitutions within the following group (Clustal W, ‘weak’ conservation group) are to be regarded as semi-conservative substitutions: -C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

Examples of conservative substitutions are substitutions made within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by Neurath and Hill (1979). The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Essential amino acids in a mammalian alpha and/or beta defensin fragment can be identified according to procedures known in the art, such as site-directed mutagenesis or alanine-scanning mutagenesis (Cunningham and Wells, 1989). See also, (Hilton et al., 1996). The identities of essential amino acids can also be inferred from analysis of identities with polypeptides which are related to mammalian alpha and/or beta defensin fragments (see Clustal W alignment in FIGS. 27-29).

Single or multiple amino acid substitutions can be made and tested using known methods of mutagenesis, recombination, and/or shuffling, followed by a relevant screening procedure, such as those disclosed by (Reidhaar-Olson and Sauer, 1988); (Bowie and Sauer, 1989); WO 95/17413; or WO 95/22625. Other methods that can be used include error-prone PCR, phage display (e.g., (Lowman et al., 1991); U.S. Pat. No. 5,223,409; WO 92/06204), and region-directed mutagenesis (Derbyshire et al., 1986); (Ner et al., 1988)). When the result of a given substitution cannot be predicted with certainty, the derivatives may be readily assayed according to the methods described herein above to determine the presence or absence of biological activity.

### Compounds/Peptides

The compounds as described herein are based on a defensin peptide fragment optionally linked via a linker to a fatty moiety. The compounds are referred to as peptides, compounds, lipopeptides, modified fragments (of defensins) and chemical modifications (of defensins).

### The Fatty Moiety (a in a-b-c)

Lipidation means herein any C- or N-terminal covalent binding of a lipid group to a mammalian (e.g. human) defensin or defensin fragment. In particular, the binding of C₄-C₂₇ long chain fatty moieties. The fatty moieties may be fatty acids such as butyric acid, lauric acid, myristic acid, palmitic acid, or stearic acid or a sterol such as cholesterol. Fatty moieties are described further herein below.

In one embodiment, the compound as defined herein is provided, wherein the fatty moiety (a) comprises at least 8 carbon atoms, such as at least 9 carbon atoms, such as at least 10 carbon atoms, such as at least 11 carbon atoms, such as at least 12 carbon atoms, such as at least 13 carbon atoms, such as at least 14 carbon atoms, such as at least 15 carbon atoms, such as at least 16 carbon atoms, such as at least 17 carbon atoms, such as at least 18 carbon atoms, such as at least 19 carbon atoms, such as at least 20 carbon atoms, such as at least 21 carbon atoms, such as at least 22 carbon atoms, such as at least 23 carbon atoms, such as at least 24 carbon atoms, such as at least 25 carbon atoms, such as at least 26 carbon atoms, such as at least 27 carbon atoms, such as at least 28 carbon atoms, such as at least 29 carbon atoms, such as at least 30 carbon atoms, such as at least 31 carbon atoms, such as at least 32 carbon atoms, such as at least 33 carbon atoms, such as at least 34 carbon atoms, such as at least 35 carbon atoms.

In one embodiment, the compound as defined herein is provided, wherein the fatty moiety (a) comprises an aliphatic chain or an aliphatic cycle.

### Aliphatic Cycles

In one embodiment, the compound as defined herein is provided, wherein the fatty moiety (a) is an aliphatic cycle comprising a gonane structure.

In one embodiment, the compound as defined herein is provided, wherein the fatty moiety (a) is a sterol. In one embodiment, the sterol is selected from the group consisting of: cholesterol, campesterol, sitosterol, stigmasterol, and ergosterol.

In one embodiment, the compound as defined herein is provided, wherein the fatty moiety (a) is an aliphatic cycle comprising a steroid, such as cholesterol.

In one embodiment, the compound as defined herein is provided, wherein the fatty moiety (a) comprises two adjacent carbon atoms connected by a double bond.

### Aliphatic Chains

In one embodiment, the compound as defined herein is provided, wherein the fatty moiety (a) is an aliphatic branched or non-branched chain.

In one embodiment, the compound as defined herein is provided, wherein the fatty moiety (a) is a non-branched C8 to C26 fatty acid, such as C12, C14, C16, C18, C20 or C22 fatty acid.

In one embodiment, the compound as defined herein is provided, wherein the fatty moiety (a) is a non-branched C16 fatty acid.

### Linker/Spacer

The insertion(s) of linker/spacer in the form of sugars, amino acid or PEG-agents can be used for further modifications. Examples of linker/spacer are glucose and/or sucrose, or cysteine, lysine or 8-amino-3.6-dioxaoctanoic acid a hydrophilic PEG agent.

### Defensin Part

The defensin part of the compound is a fragment of a defensin. Examples of fragments of defensins can be found in tables in Example 14. Preferably, the fragment is a fragment that has been identified by mass spectrometry. Suitable examples of the peptide component of the compounds include SEQ ID NO: 6, 36, and 39, which are fragments of hBD1, SEQ ID NO: 93, 93, 94, 97, 99, 103, 105, 106, 114, 119, and 122, which are fragments of HDS, and SEQ ID NO: 181, 189, 190, 192, 195, 198, 203, and 206, which are fragments of HNP4. Preferably, the peptide is a fragment of hBD1 or HD5, more preferably a fragment of hBD1 selected from SEQ ID NO: 36, and 39. The five Pams described herein are based on SEQ ID NO: 36.

### Methods and Uses

Previous studies have demonstrated an enhanced activity of AMPs after fatty acid modification at the N-terminal end. The inventors selected a C₁₆ long fatty acid, palmitic acid, and tested it with different spacers such as sugars or amino acids to improve the stability and bactericidal activity of an hBD-1 derived octapeptide while preserving the host's microbiota. Five unique lipopeptides, Pam-1, Pam-2, Pam-3, Pam-4 and Pam-5, were designed based on the octapeptide generated from reduction of hBD-1 (FIG. 1). Pam-2 represents the precursor for all other Pams, the N-terminal palmitoylated octapeptide. Pam-3 additionally contains 8-Amino-3.6-dioxaoctanoic acid, a PEG-agent as hydrophilic spacer. While Pam-1 contains glucose and sucrose as spacer, Pam-4 contains cysteine and Pam-5 lysine. The idea behind the concept of different spacers is to change the structure, which could lead to various mechanisms of action.

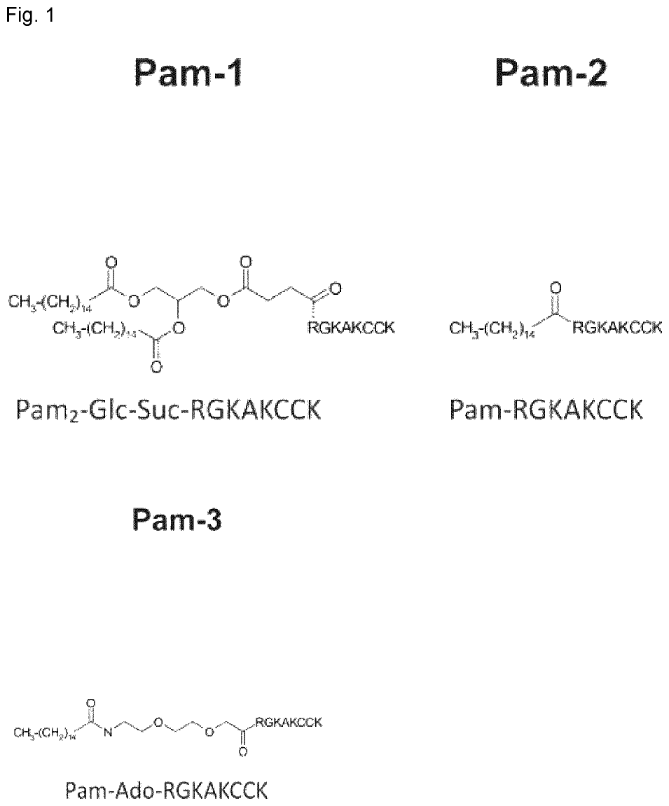

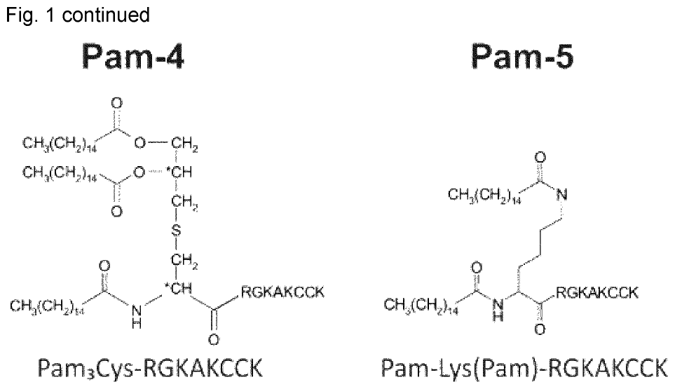

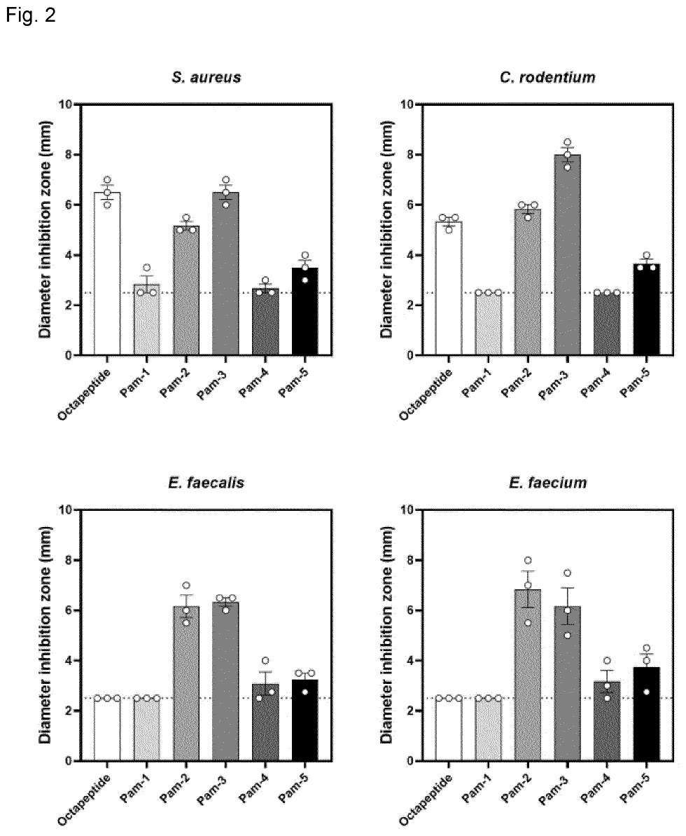

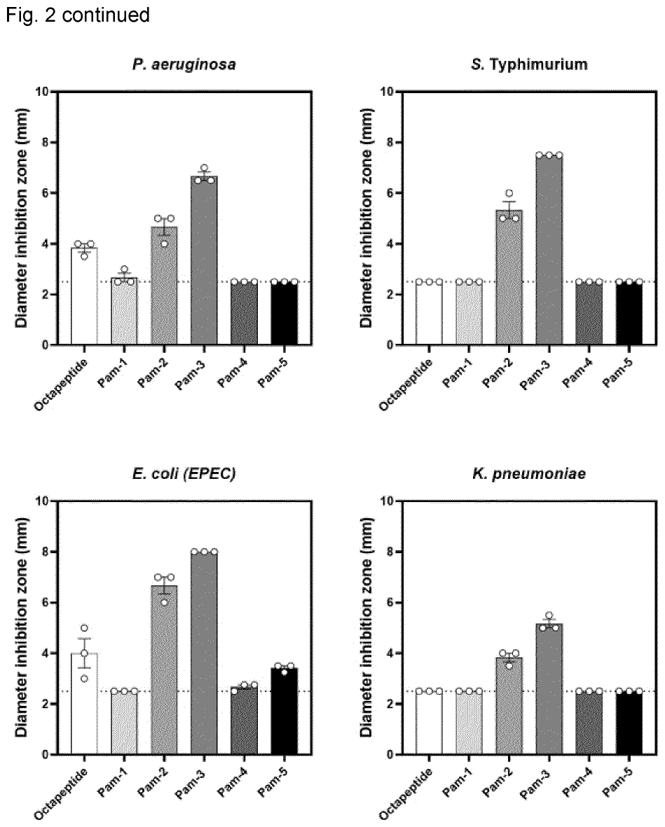

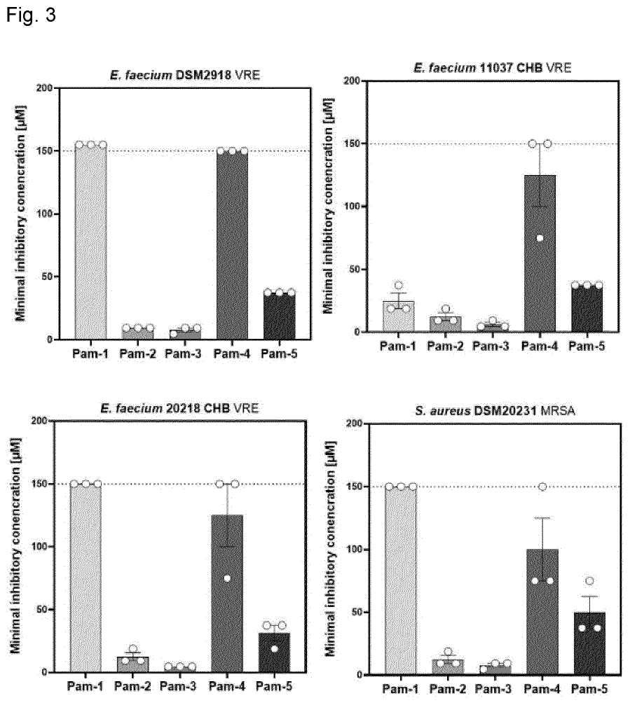

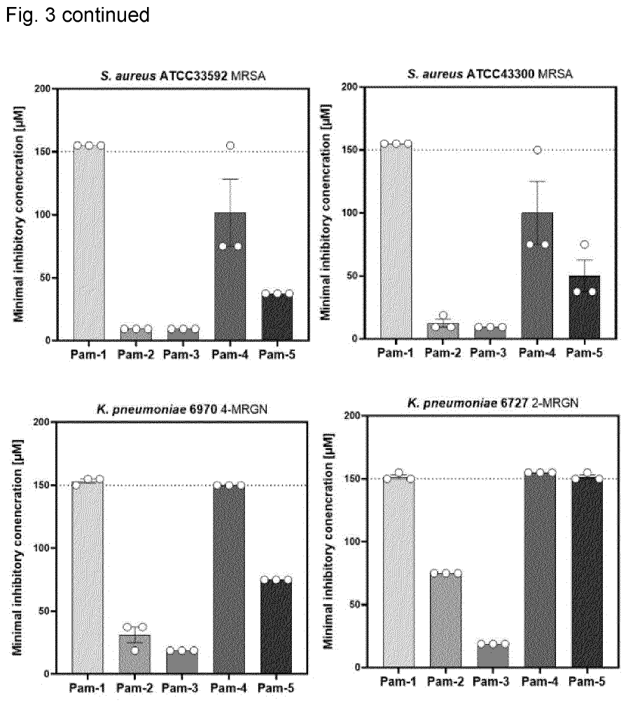

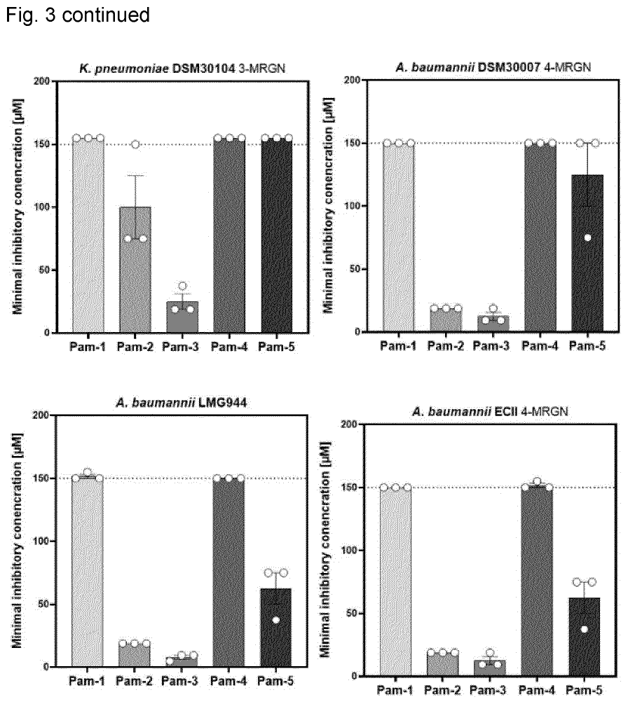

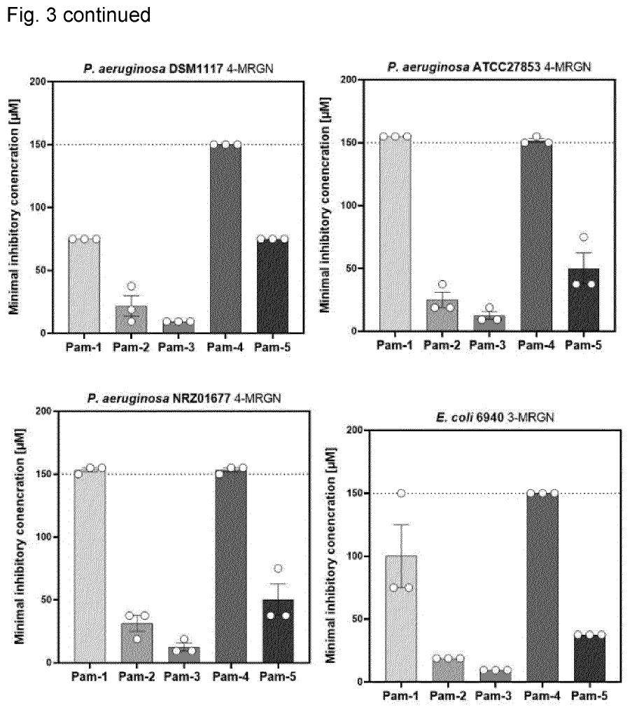

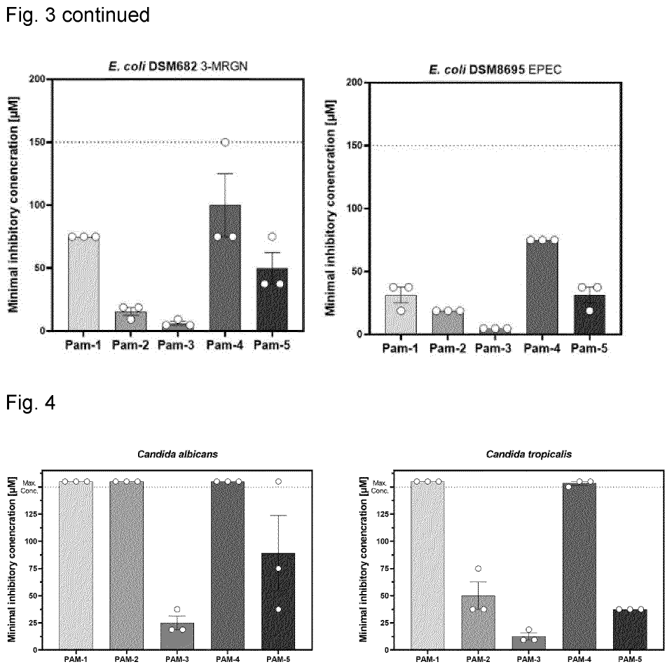

Surprisingly Pam-1, Pam-4 and Pam-5 were generally inactive against the tested bacterial strains, strongly contrasting the potent inhibition of bacterial growth mediated by Pam-2 and Pam-3 (FIG. 2, FIG. 3 and FIG. 25). Bacterial growth was most strongly inhibited by Pam-3, either on par with (S. aureus) or superior (C. rodentium, P. aeruginosa and S. Typhimurium) to the octapeptide, pointing towards modification-specific activities. Notably, both Pam-2 and Pam-3 consistently inhibited S. Typhimurium growth, a species the non-modified octapeptide failed to inhibit. Pam-3 was also highly effective in vitro against A. baumannii resistant to the last-resort antibiotics, colistin and tigecycline (FIG. 3 and FIG. 25). Pam-2, Pam-3 and Pam-5 were highly effective against C. tropicalis, whereas only Pam-3 was highly effective and Pam-5 showed some efficacy against C. albicans (FIG. 4).

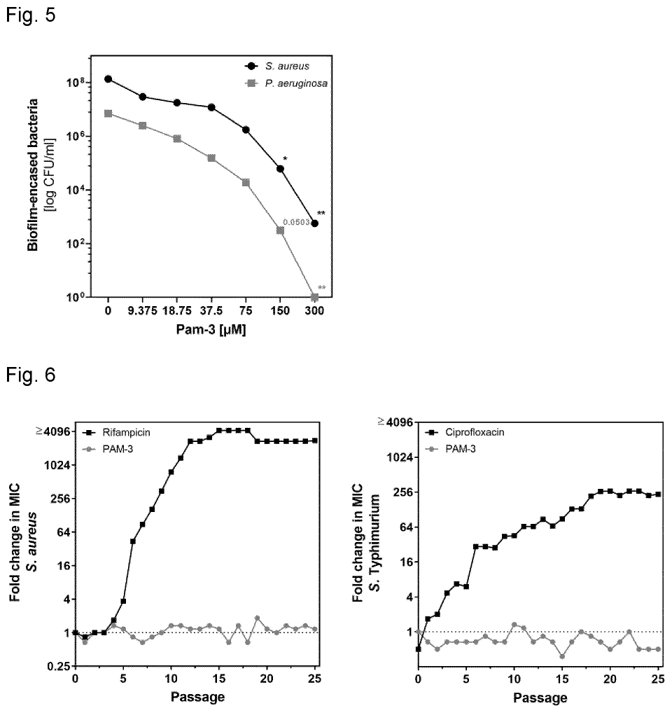

Bacterial biofilms are highly resistant to growth inhibitors and bactericidal treatment regimens. Apart from the hindered penetration of antibacterial agents, treatment is further complicated by 10 to 1000 times increased tolerance exhibited by biofilm protected bacteria compared to planktonic bacteria. The inventors demonstrated the ability of Pam-3 to eradicate established biofilms in a dose-dependent manner. Within 1 hour, 300 μM of Pam-3 eliminated P. aeruginosa biofilms and similarly eradicated ˜99.99% of S. aureus in biofilms (FIG. 5).

Development of antibiotic resistance is increasing at an alarming rate. The ability of Gram positive S. aureus and Gram negative S. Typhimurium to develop resistance against Pam-3 was assessed. When cultured in the presence of sub-inhibitory concentrations of Pam-3 for 25 passages, no significant increase in the minimal inhibition concentration (MIC) was observed for S. aureus. In contrast, the MIC for the standard antibiotic, rifampicin, started to rapidly increase after five passages and had increased ≥4096-fold after 15 passages (FIG. 6). Similarly, although exposed to Pam-3 for continuous serial passages, no resistant S. Typhimurium isolates emerged, whereas the presence of ciprofloxacin, resulted in an increased MIC already after 3 passages, and a ≥256-fold MIC increase after 19 passages (FIG. 6). These results indicate that resistance development against Pam-3 is negligible, if at all present, thus highly contrasting the emerging resistance to conventional antibiotics.

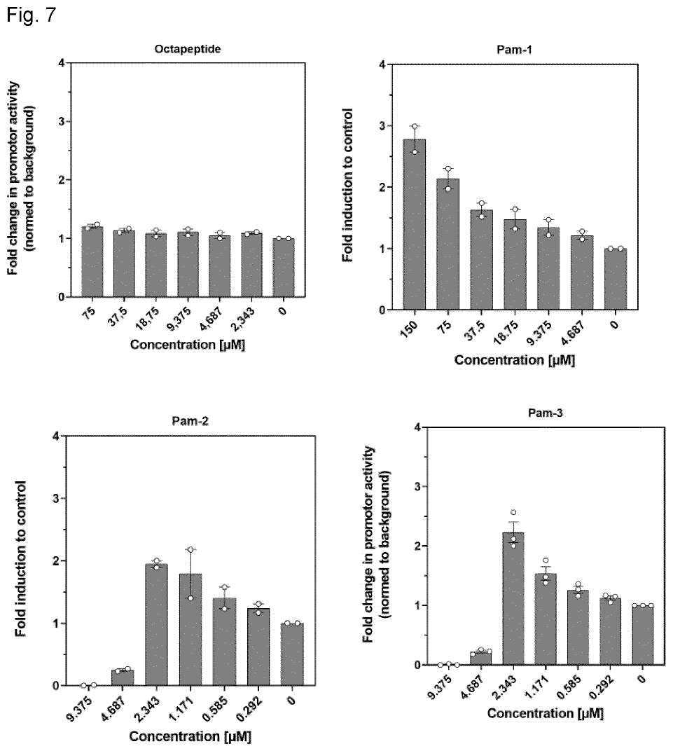

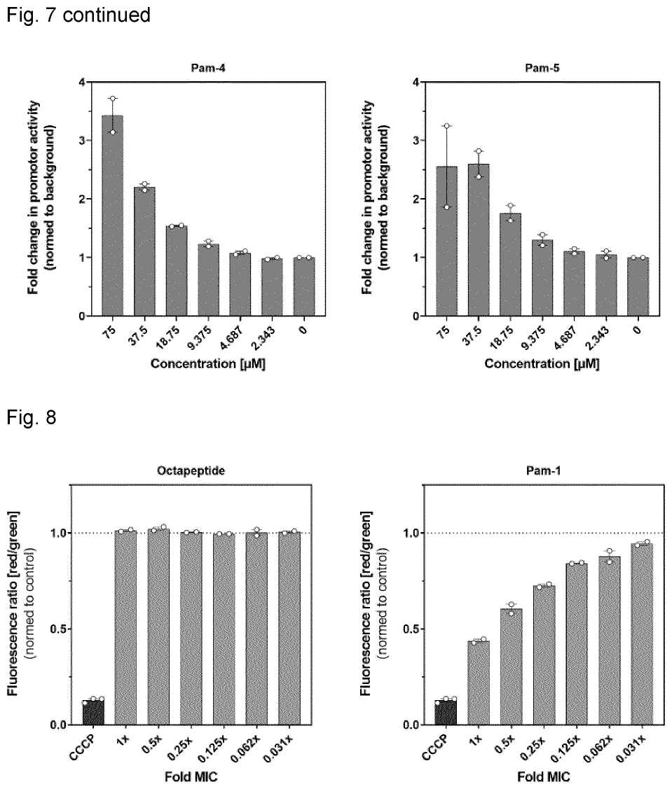

A primary target of antimicrobial peptides is the bacterial cell envelope. Disturbing the integrity and function of the outer and/or inner membranes result in loss of the barrier function and dissipation of the membrane potential (Cole and Nizet, 2016). To clarify the mode of action of the octapeptide derived lipopeptides, the inventors used a ypuA promotor-based luciferase reporter strain of B. subtilis to identify cytoplasmic membrane-associated and cell envelope-related stress. The ypuA promoter was activated (2-fold) by Pam-2 and Pam-3, indicating cell envelope impairment whereas Pam-1, Pam-4 and Pam-5 surprisingly seemed to invoke little cell envelope damage (FIG. 7).

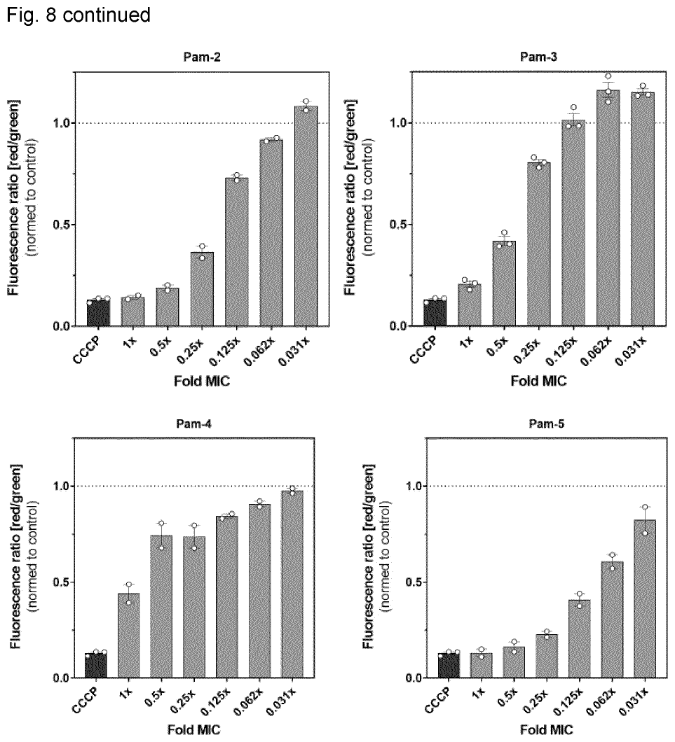

The influence of the octapeptide derived lipopeptides on the transmembrane potential of S. aureus NCTC8325 was also assessed. The protonophore carbonyl cyanide m-chlorophenyl hydrazone (CCCP) was used as a positive control to depolarize bacteria, i.e. leading to a reduction of their membrane potential. Upon depolarization, DiOC₂(3) shifts from green fluorescence towards red emission because of self-association of the dye molecules. Treatment with any of the lipopeptides caused a breakdown of the membrane potential in a concentration-dependent manner but was most pronounced for Pam-2, Pam-3 and Pam-5 (FIG. 8). Both results emphasize that the octapeptide derived lipopeptides acts on bacterial membranes.

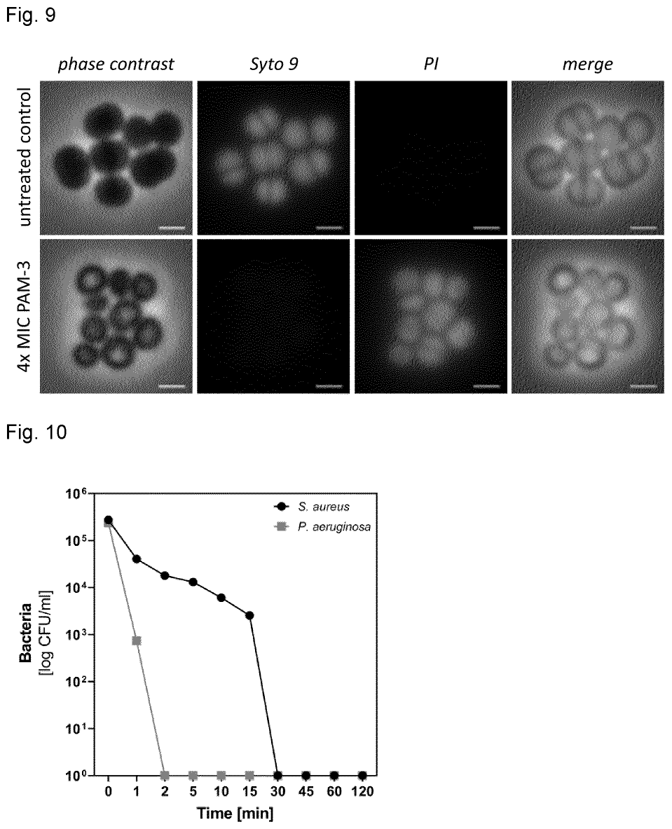

Since a large variety of AMPs target the membrane as pore formers, the ability of Pam-3 to induce membrane lesions was analyzed. S. aureus NCTC8325 was treated with Pam-3 at 4× MIC and added a mixture of Syto9 and propidium iodide (PI). The membrane-permeant Syto9 stains all living cells green, whereas the red-fluorescent PI can only enter cells through large membrane pores or lesions. Pam-3 led to a strong influx of PI (FIG. 9). Pore formation was associated with fast killing of these bacteria. The bactericidal kinetics i.e. the rapidness of Pam-3 mediated killing was assessed. Pam-3 killed more than 90% of P. aeruginosa within 1 min and S. aureus within 15 min (FIG. 10). Eradication to the level of detection was observed 2 and 30 min after Pam-3 treatment, respectively.

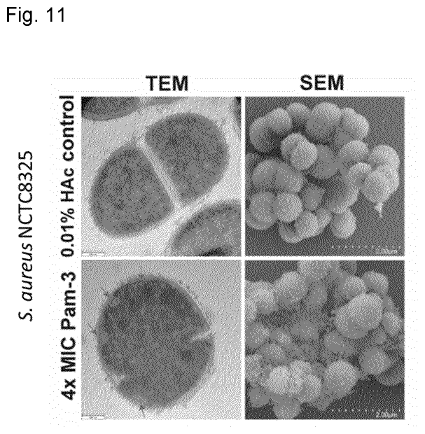

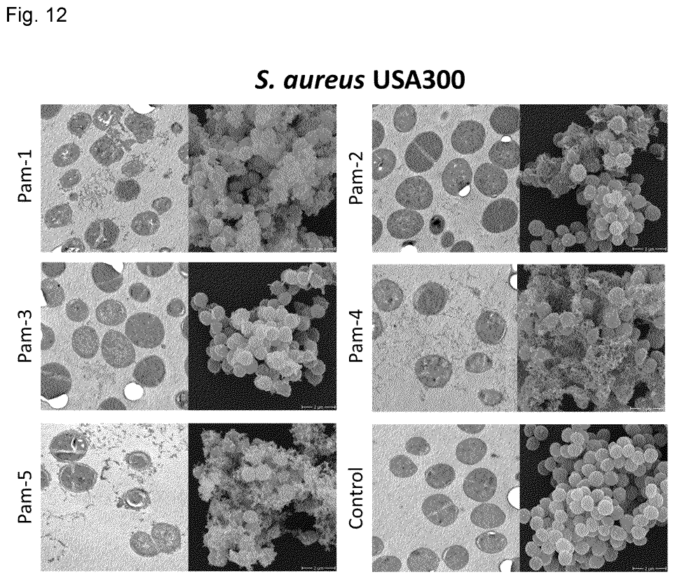

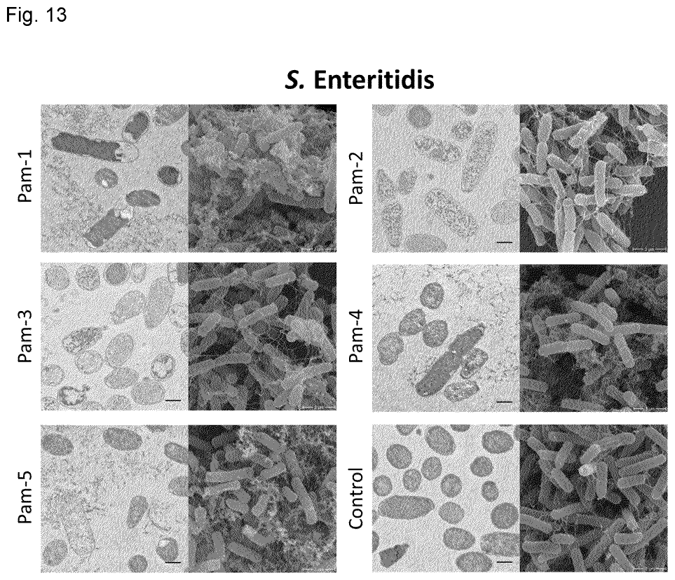

Scanning electron microscopy (SEM) was employed to observe cell morphological changes after Pam-3 treatment directly. Exposure to Pam-3 resulted in membrane surface disruption and lysed cells similar to hBD1, while control cells exhibited a bright and smooth surface (FIG. 11). Transmission electron microscopy (TEM) was performed to analyze changes in bacterial morphology to compare bacterial morphology before and after treatment with the five lipopeptides in S. aureus and S. Enteritidis. In agreement with the pore formation and the induction of cell envelope stress and depolarization, treatment with octapeptide derived lipopeptides resulted in strong cell envelope damage with disrupted membranes and pores in most of the cells (FIGS. 12 and 13).

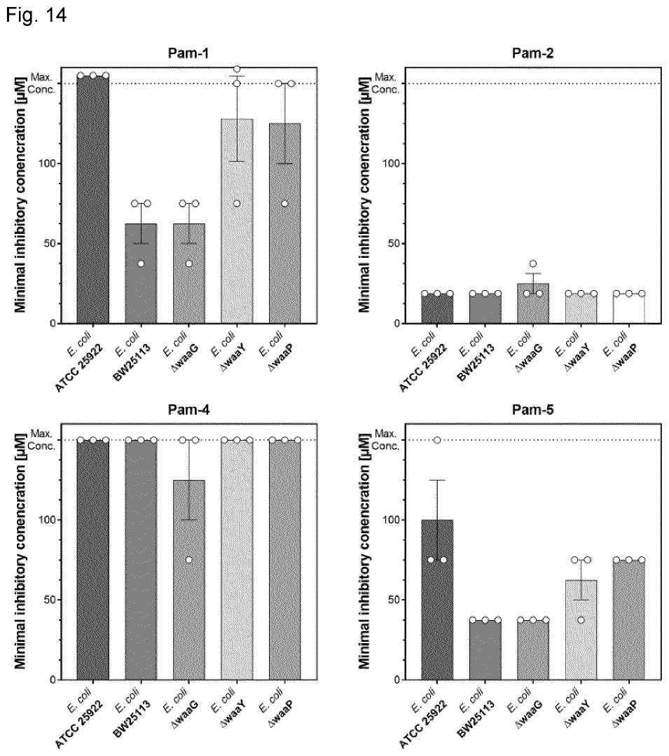

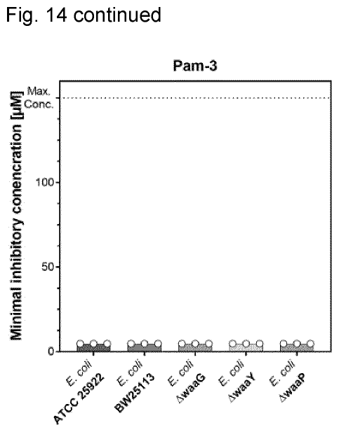

Additionally, E. coli BW 25113 mutants differ in their LPS composition which makes them useful to study the binding of Pams to Gram negative bacteria. For comparison, E. coli BW 25113 and E. coli ATCC 25922 were utilized as control. E. coli ATCC 25922 possess the full-length LPS thus additional an O-antigen while E. coli BW 25113 lacks the O-antigen. The E. coli BW 25113 mutant ΔwaaG has similar phosphate residues like the wild type thus the same charge but is lacking an outer core. The mutant ΔwaaY possess an outer core while lacking one phosphate residue in the inner core leading to a less negative charge of the cell wall. A similar LPS composition has the mutant ΔwaaP except the amount of phosphate residues in the inner core which is zero (FIG. 14).

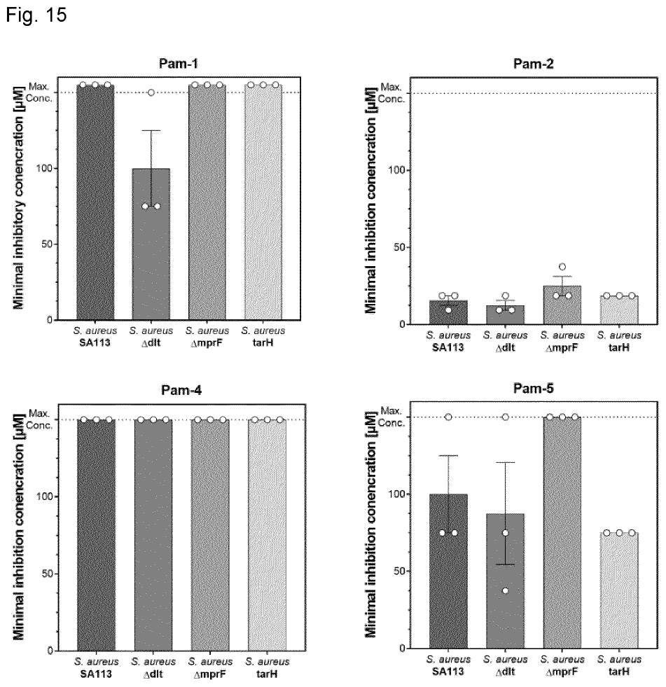

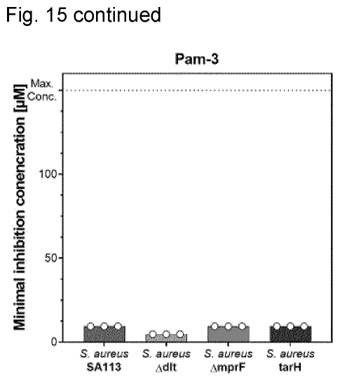

In order to identify cell wall targets of Gram positive bacteria, cell wall mutants of S. aureus SA113 were investigated regarding their susceptibility against the Pams. S. aureus SA113 was used as control. The ΔdItA mutant lacks D-alanine in the peptidoglycan layer resulting in a more negative charge which should facilitate the binding of Pams and subsequent cell wall disruption (VVeidenmaier et al., 2005). A similar cell wall composition has the ΔmprF mutant lacking L-lysin in the cell membrane resulting in a more negative charge (Peschel and Collins, 2001). Additional teichoic acid in the peptidoglycan layer possess the tarH mutant causing a strengthening of it (FIG. 15) (Wanner et al., 2017).

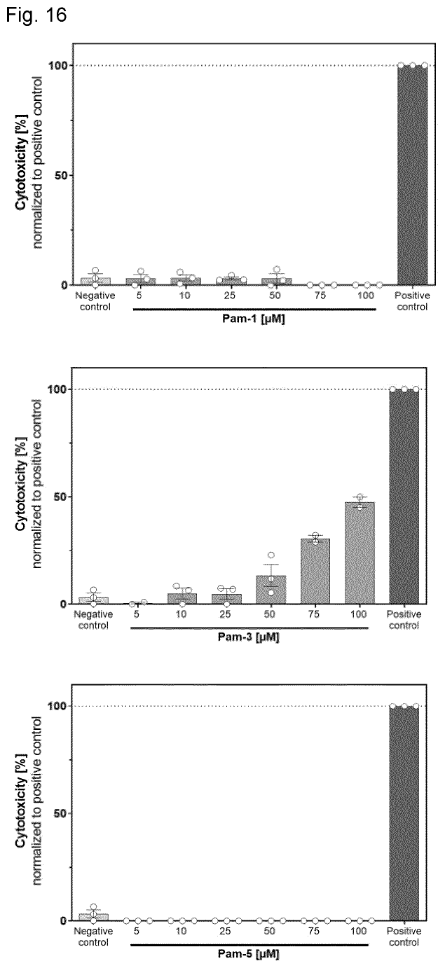

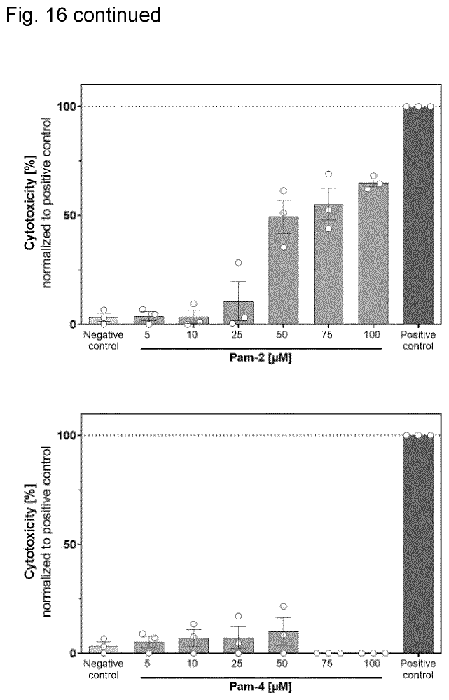

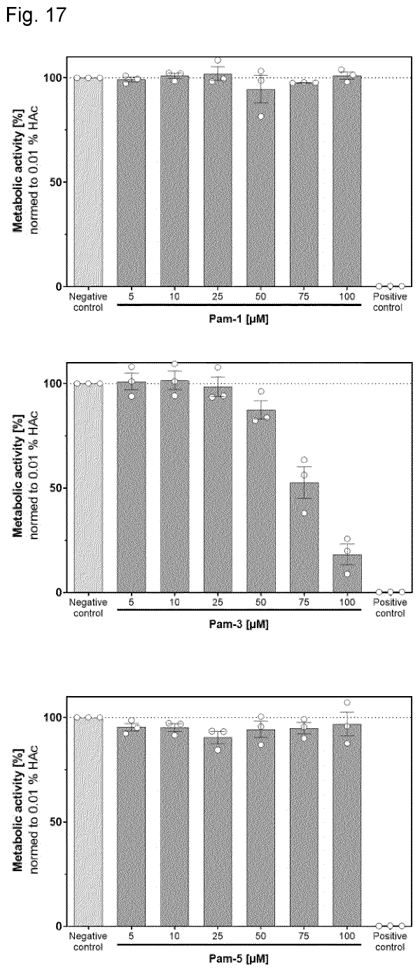

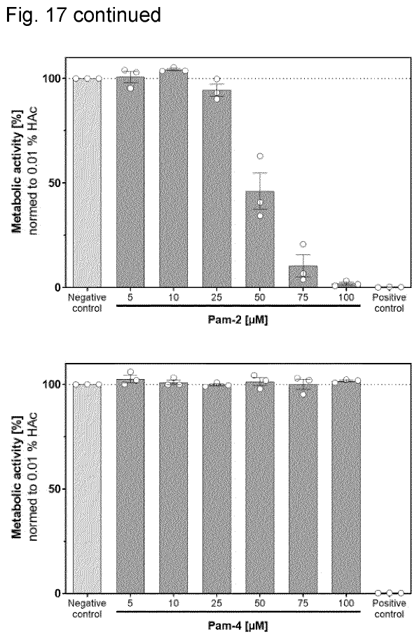

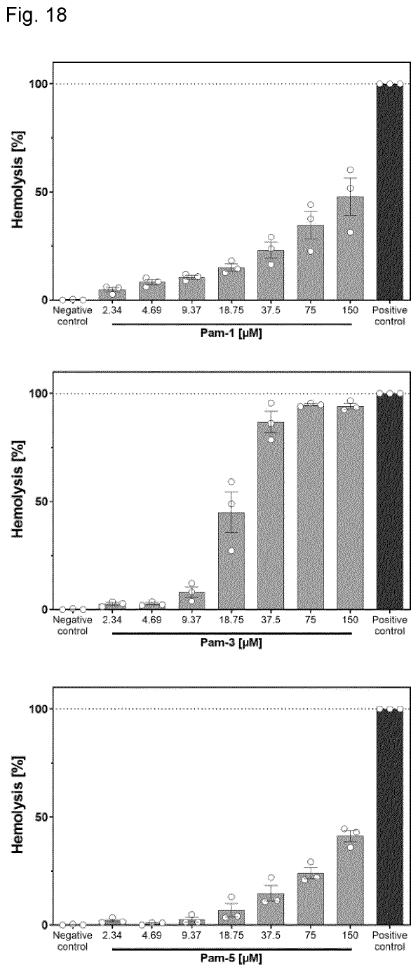

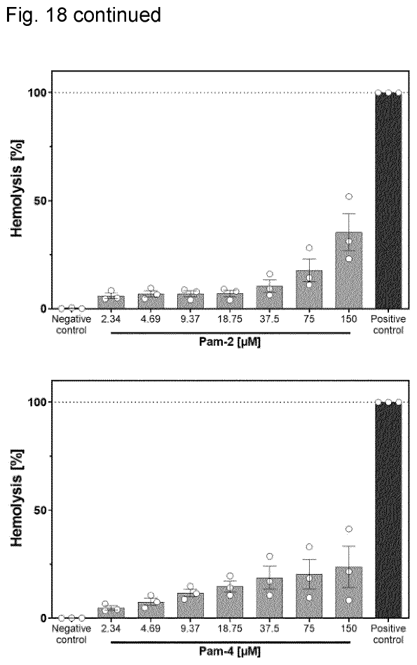

The inventors further demonstrated that the antimicrobial activity of Pams is independent of LPS and bacterial cell wall charge (FIGS. 14 and 15). High antimicrobial efficacy is commonly associated with low tolerability and AMP's are no exception. Tolerability of the octapeptide derived lipopeptides was assessed employing LDH, WST-1 and hemolysis assays. Pam-1, Pam-4 and Pam-5 were non-toxic and displayed no LDH activity. Pam-3 showed little LDH activity and Pam-2 showed some LDH activity (FIG. 16). Pam-1, Pam-4 and Pam-5 also displayed normal metabolic activity as assessed by WST-1 assay. Pam-3 showed a slight decrease and Pam-2 a modest decrease of metabolic activity (FIG. 17). Pam-1, Pam-2, Pam-4 and Pam-5 showed minimal red blood cell disruption whereas Pam-3 showed modest red blood cell disruption (FIG. 18).

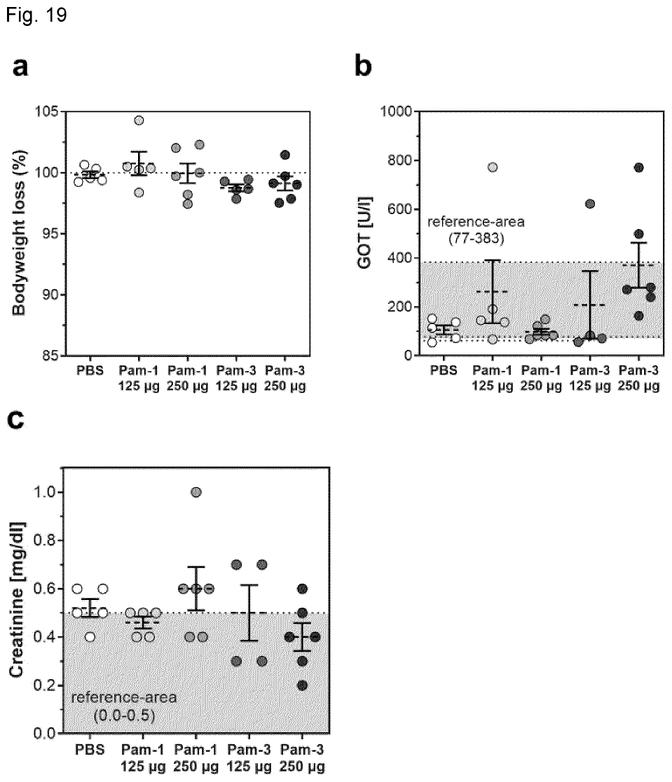

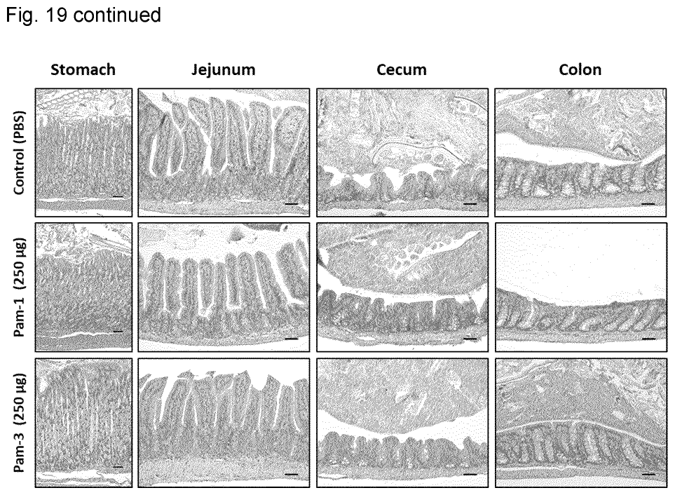

Assessment of acute tolerability of Pams was assessed after oral administration of Pam-1 and Pam-3 in mice. Histological analysis and determination of serum markers 24 hours after application of two doses of 125 μg or one dose of 250 μg of Pam-1 or Pam-3 respectively did not reveal any acute toxicity. Specifically, there were no alterations in bodyweight and no signs of systemic toxicity or distress (FIG. 19). Measurement of serum levels of creatinine and glutamic oxaloacetic transaminase showed no significant differences between the groups suggesting no effect on kidney and liver metabolism (FIG. 19). Finally, histological examination of gastrointestinal, liver and kidney tissues revealed no alterations, except a minor shortening of intestinal villi of the jejunum-ileum of one control and two treated mice (FIG. 19). As no difference in these histopathological findings was observed between PBS and Pam-3 treated animals, they were regarded as background observations (FIG. 26). It was thus concluded that despite slight differences in tolerability as determined by cytotoxic assays surprisingly neither Pam-1 nor Pam-3 treatment was associated with acute toxicity.

In vivo efficacy of Pam-1 and Pam-3 was tested in an acute gastrointestinal infection caused by S. Typhimurium and an established gastrointestinal infection caused by C. rodentium.

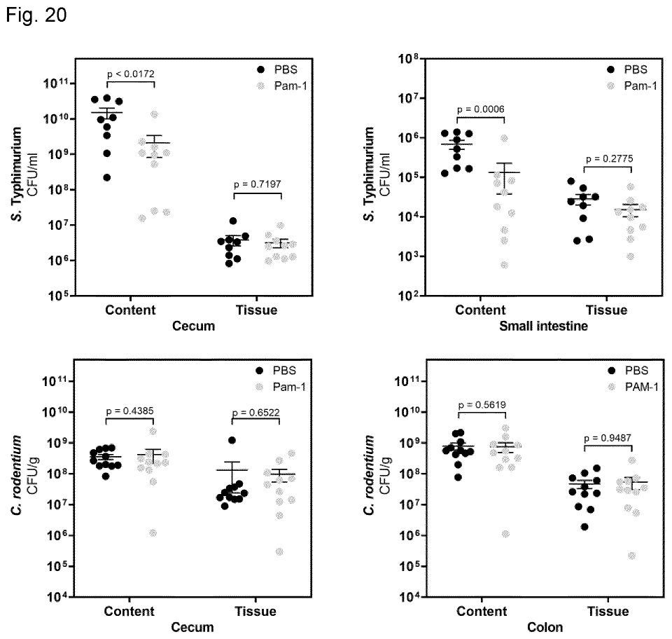

Pam-1 treated animals showed significantly reduced Colony Forming Units (CFU) of S. Typhimurium in cecum content but not in tissue (Mann-Whitney test, p<0.017 and p=0.7197, respectively, FIG. 20). Pam-3 treated animals showed significantly reduced CFU's of S. Typhimurium in cecum content as well as in cecum tissue (Mann-Whitney test, p<0.0001 and p=0.0409, FIG. 20). Furthermore, Pam-3 also lowered the pathogenic load in the small intestine without affecting the small intestine tissue (Mann-Whitney test, p=0.0024 and p=0.8621, respectively, FIG. 20).

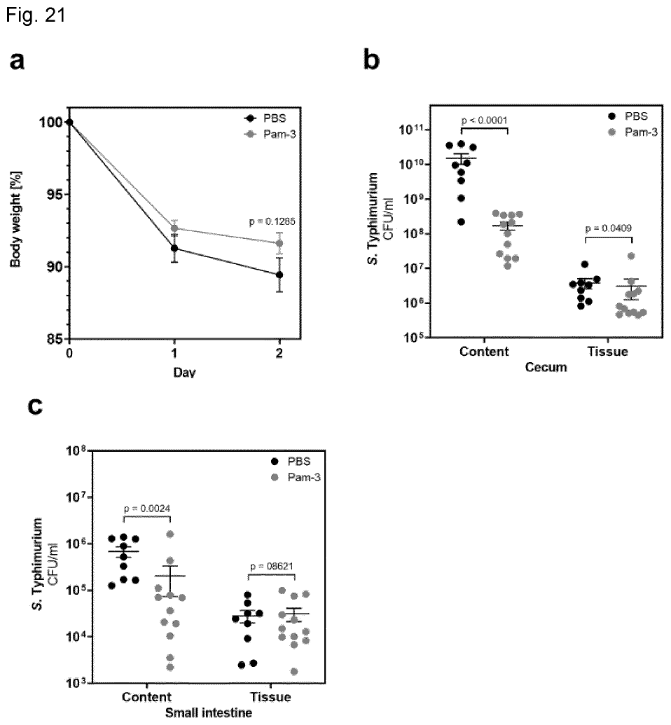

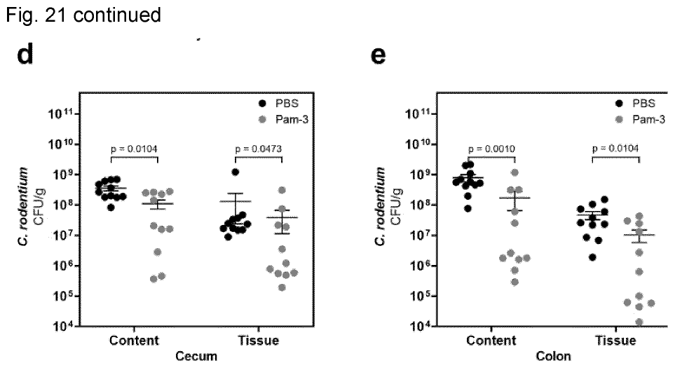

Pam-1 treatment of mice infected with C. rodentium surprisingly did not reduce CFU's in cecum nor in colon (FIG. 20). Pam-3 treatment reduced the number of C. rodentium in the cecum content and cecum tissue (Mann-Whitney test, p=0.0104 and p=0.0473, FIG. 21). Treatment also significantly reduced the number of viable Citrobacter in colon content and colon tissue (Mann-Whitney test, p=0.0010 and p=0.0104).

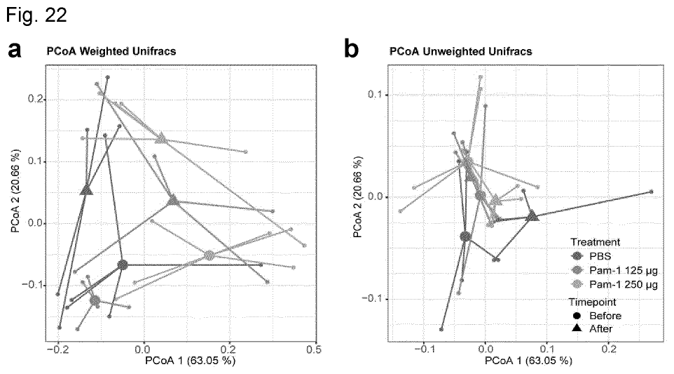

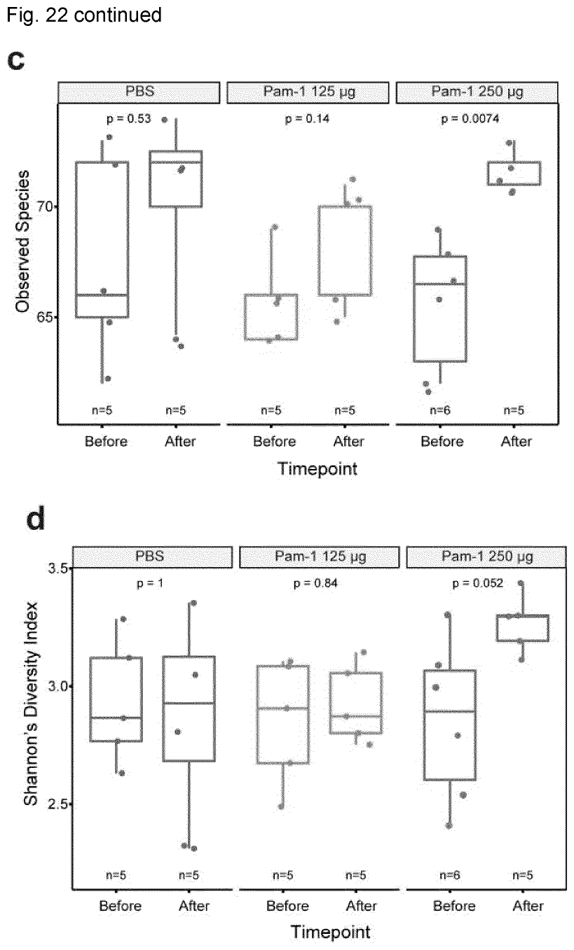

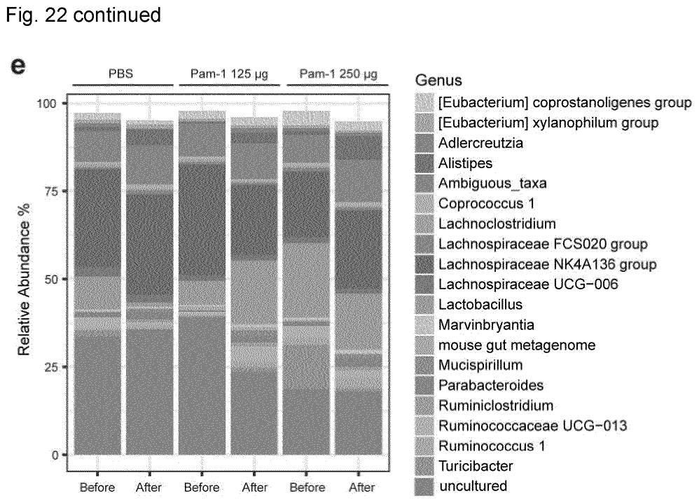

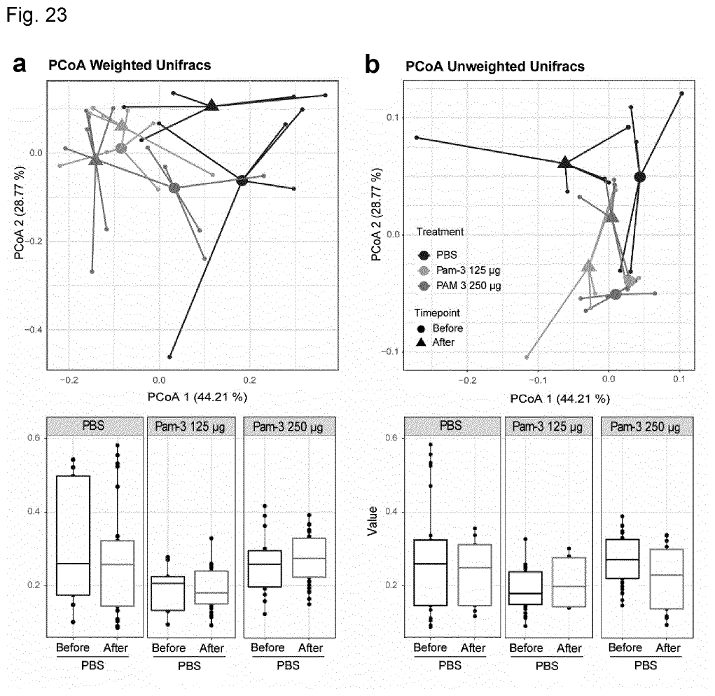

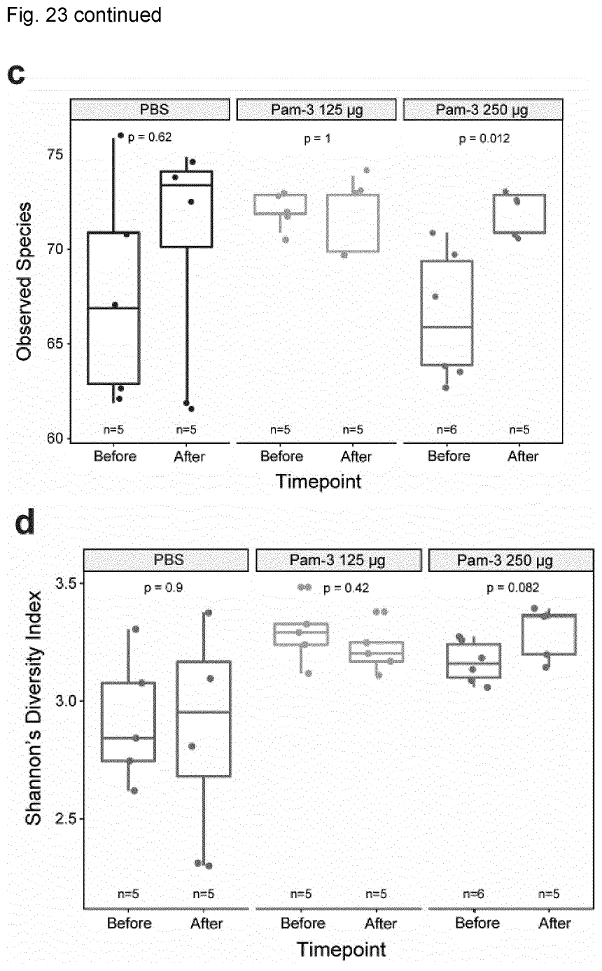

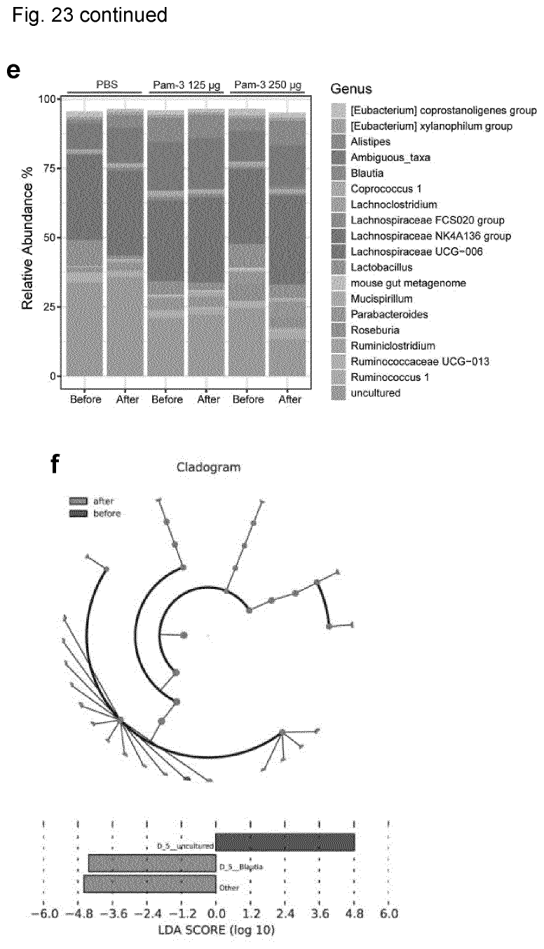

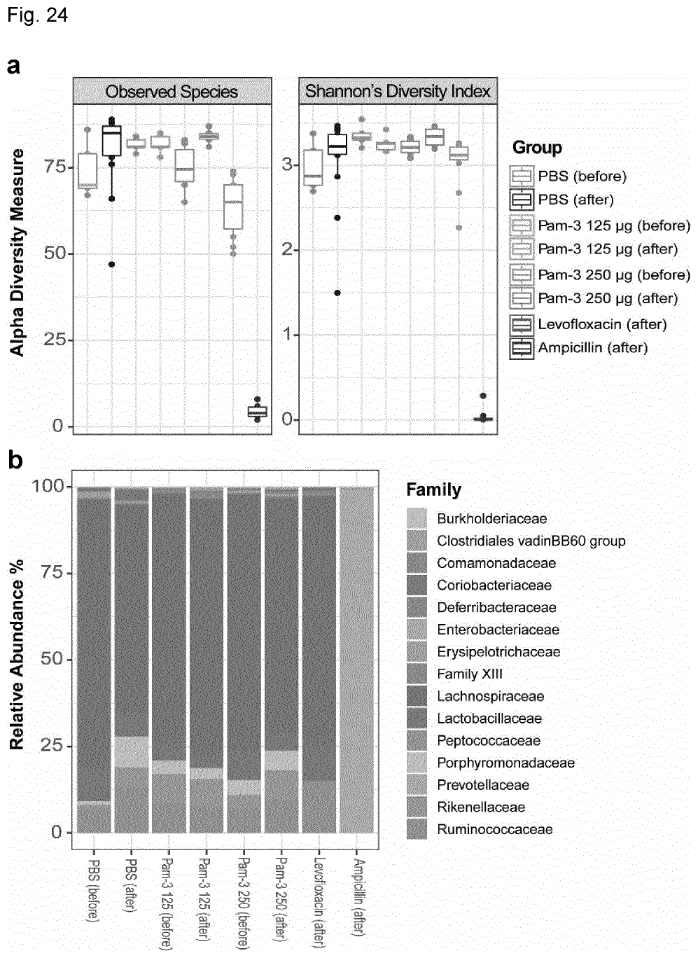

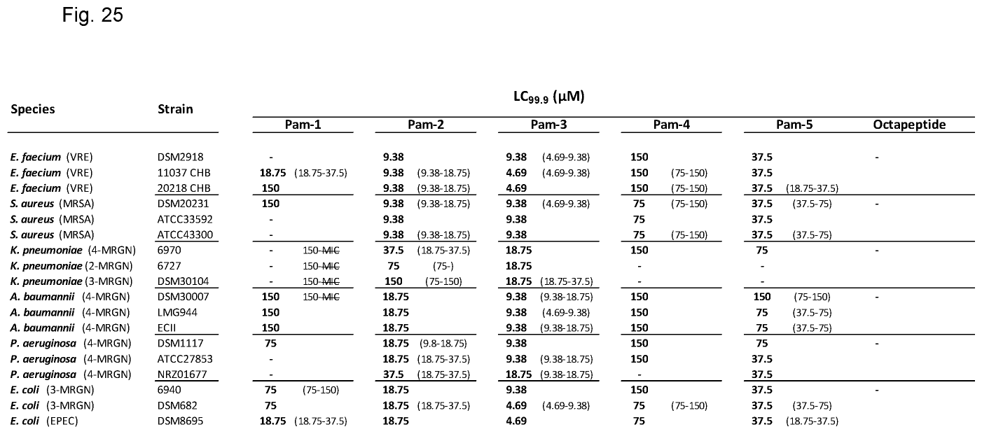

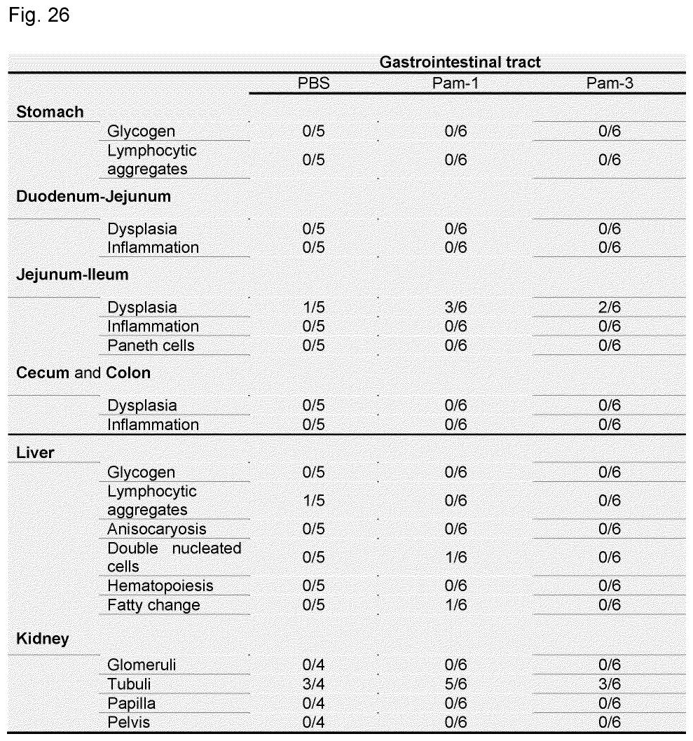

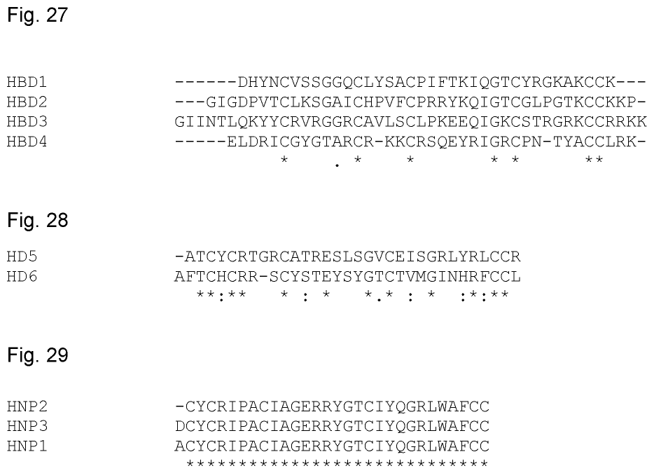

Antibiotic treatment has notable consequences. Observational, clinical, and epidemiologic studies have demonstrated that antibiotic treatment affects the gut microbiota composition with immediate effects on health (Blaser, 2016; Francino, 2016). Changes in the microbiota composition, decreased diversity, reduced taxonomic richness and dysbiosis are the main consequences (Dethlefsen and Relman, 2011; Dethlefsen et al., 2008). Further, antibiotics can have long-term effects such as increased susceptibility to infections, obesity and obesity-associated metabolic diseases (Francino, 2016; Lange et al., 2016). In contrast to conventional antibiotics and surprisingly when considering their antimicrobial efficacy, neither Pam-1 nor Pam-3 treatment had any appreciable effects on commensal microbes. Analysis of beta diversity and calculation of weighted Unifrac Distances demonstrated that changes in the microbiota between the before and after samples were similar between Pam-1 and Pam-3 treated mice and PBS gavaged control mice (FIGS. 22 & 23). Similarly, while minor changes in the community structure were observed in both groups (i.e. treated and untreated), the number of detected species as well as the complexity (FIGS. 22 & 23) remained comparable, thus contrasting treatment with traditional antibiotics, such as ampicillin (FIG. 23). In line with the analysis of alpha and beta diversity, neither Pam-1 nor Pam-3 treatment affected the abundance of bacterial genera (FIGS. 22 & 23). Combined, these results surprisingly demonstrate that, neither Pam-1 nor Pam-3 are associated with notable effects on the healthy microbiome. These data demonstrate that treatment with Pams would be a significant advantage over conventional antibiotics where a disruptive effect on the resident microbiota as well as a rapid drop in diversity is commonly observed (Burdet et al., 2019). Furthermore, after antibiotic treatment, the intestine is often colonized by non-commensal bacteria, which can result in long-term environmental changes (de Lastours and Fantin, 2015). Instead of a loss of diversity, even high-dose treatment with the potent antimicrobial Pam-3 showed an unaffected bacterial diversity.

In conclusion, the inventors demonstrate that octapeptide derived lipopeptides and in particular Pam-2 and Pam-3 are promising alternatives to fight multidrug resistant infections in a post-antibiotic world because of their broad antimicrobial activity against Gram-positive and Gram-negative pathogens as well fungi and their efficacy against gastrointestinal infections importantly without disrupting the resident microbiota.

### In Vitro Synthesis

Mammalian defensin fragments and mammalian defensin fragments coupled with lipopeptides may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms) e.g. D-alanine and D-isoleucine, diastereoisomers, side chains having different lengths or functionalities, and the like. The particular sequence and the manner of preparation will be determined by convenience, economics, purity required, and the like. Chemical linking may be provided to various peptides or proteins comprising convenient functionalities for bonding, such as amino groups for amide or substituted amine formation, e.g. reductive amination, thiol groups for thioether or disulphide formation, carboxyl groups for amide formation, or lipidation and the like. If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

### Dosages

A mammalian alpha or beta defensin fragment coupled with lipopeptides to generate a stable peptide with antimicrobial properties preferably employed in pharmaceutical compositions in an amount which is effective to prevent or treat an infection caused by Gram positive or Gram negative bacteria, viruses, protozoae, fungi or helminths while preserving the host microbiota.

For such treatments, the appropriate dosage will, of course, vary depending upon, for example, the chemical nature and the pharmacokinetic data of a compound used, the individual host, the mode of administration and the nature and severity of the conditions being treated.

However, in general, for satisfactory results in mammals, for example humans, an indicated daily dosage of a human alpha defensin or beta defensin derived fragment coupled with a lipopeptide (Pam) is preferably from about 0.1 mg Pam/kg body weight to about 1000 mg Pam/kg body weight, more preferably from about 1.0 mg Pam/kg body weight to about 500 mg Pam/kg body weight, for gexample, administered in divided doses up to one, two or three times a day or continuously.

The compounds of preferred embodiments can be administered to mammals, for example humans, by similar modes of administration at similar dosages than conventionally used.

Appropriate concentrations and dosages can be readily determined by one skilled in the art.

In one embodiment, the mammalian Pam is administered at least once daily, such as at least twice daily, for example at least 3 times daily or continuously.

### Formulations for Oral or Parenteral Administration

Mammalian alpha defensin fragments and beta defensin fragments coupled with a lipopeptide to generate a Pam can be employed therapeutically in compositions formulated for administration by any conventional route.

In one embodiment, the administration of at least one Pam according to the disclosed methods is oral.

In one embodiment, the administration of at least one Pam, according to the disclosed methods, is generally intranasal or intrapulmonary. Intranasal and intrapulmonary administration is normal for pulmonary drug delivery.

In one embodiment, the administration of at least one mammalian Pam according to the disclosed methods is subcutaneous or intravenous.

Within some embodiments, compositions of preferred embodiments may be formulized as a lyophilizate, utilizing appropriate excipients that provide stability as a lyophilizate, and subsequent to rehydration. Pharmaceutical compositions containing a mammalian Pam can be manufactured according to conventional methods, e.g., by mixing, granulating, coating, dissolving or lyophilizing processes. In a preferred embodiment, pharmaceutical compositions containing a mammalian Pam are formulated as a sterile and isotonic solution.

Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats, and other common additives.

The disclosed compounds may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, toothpaste, gel dentrifrice, chewing gum, or solid form preparations which are intended to be converted shortly before use to liquid form preparations, such as solutions, suspensions, and emulsions.

The disclosed compounds may be formulated in a wide variety of formulations for buccal, sublingual, oral, rectal, vaginal, dermal, transdermal, intracranial, subcutaneous or intravenous administration. The formulation can contain (in addition to a mammalian alpha defensin fragment and/or a mammalian beta defensin fragment and other optional active ingredients) carriers, fillers, disintegrators, flow conditioners, sugars and sweeteners, fragrances, preservatives, stabilizers, wetting agents, emulsifiers, solubilizers, salts for regulating osmotic pressure, buffers, diluents, dispersing and surface-active agents, binders, lubricants, and/or other pharmaceutical excipients as are known in the art. One skilled in this art may further formulate mammalian Pams in an appropriate manner, and in accordance with accepted practices, such as those described in Remington's Pharmaceutical Sciences, Gennaro (1990).

### EXAMPLES

### Example 1

### Screening of Octapeptide Based Lipopeptides for Antimicrobial Activity

Methods: Log-phase bacteria were cultivated for up to 18 hours in TSB (TSB, Becton Dickinson, USA), washed and diluted to 4×106 CFU in 10 ml agar. Bacteria were incubated in 10 ml of 10 mM sodium phosphate, pH 7.4, containing 0.3 mg/ml of TSB powder and 1% (w/v) low EEO-agarose (AppliChem). Lipopeptides were pipetted into punched wells and diffused into the gel for 3 hours at 37° C. After that, a nutrient-rich gel with 6% TSB (w/v) and 1% agarose in 10 mM sodium phosphate buffer was poured on top of the first gel and incubated for up to 24 hours at 37° C. Then the diameter of inhibition zones was measured.

Clinical isolates of A. baumannii DSM30007, E. faecium DSM2918, K. pneumoniae DSM30104 and P. aeruginosa DSM1117 were provided by the Department for Laboratory Medicine at Robert-Bosch-Hospital Stuttgart, Germany. C. rodentium DSM16636 and E. coli DSM8695 were obtained from the Deutsche Sammlung von Mikroorganismen and Zellkultur GmbH Braunschweig, Germany. Clinical isolates of A. baumannii LMG944, A. baumannii ECII, E. coli 6940, E. coli DSM682, E. faecium 11037 CHB, E. faecium 20218 CHB, K. pneumoniae 6727 and K. pneumoniae 6970 as well as S. aureus DSM20231, S. aureus ATCC25923, S. aureus ATCC33592, S. aureus ATCC43300, S. enterica serovar Typhimurium DSM554, P. aeruginosa ATCC27853, P. aeruginosa NRZ01677 and P. aeruginosa PAO1 were provided by the Institute of Medical Microbiology and Hygiene Tübingen, Germany. B. subtilis ypuA and S. aureus NCTC8325 were obtained from the Interfaculty Institute for Microbiology and Infection Medicine, Tübingen, Germany. The wild-type S. Typhimurium strain SL1344 harboring a chromosomally integrated luxCDABE cassette, which is confirmed by kanamycin resistance and the nalidixic acid and kanamycin-resistant, bioluminescent C. rodentium strain ICC180 were obtained from Helmholtz Centre for Infection Research Braunschweig, Germany.

All bacteria were stored in cryo vials (Roth) at −80° C. Before each experiment, inoculae from the frozen stocks were grown overnight at 37° C. on LB or Columbia blood agar plates (BD). For experiments, fresh cultures were prepared in tryptic soy broth (BD). All lipopeptides were chemically synthesized by EMC Microcollections GmbH (Tübingen, Germany) and purified by precipitation. All peptides were dissolved in 0.01% acetic acid.

Results: The five octapeptide derived Pams (lipopeptides) (FIG. 1) were screened for their antimicrobial activity against several pathogenic bacteria using a radial diffusion assay (FIG. 2) and MIC determinations (FIG. 3). Pam-1, Pam-4 and Pam-5 were generally inactive against the tested strains, strongly contrasting the surprising potent inhibition of bacterial growth mediated by Pam-2 and Pam-3. Bacterial growth was most strongly inhibited by Pam-3, either on par with (S. aureus) or superior (C. rodentium, P. aeruginosa and S. Typhimurium) to the octapeptide, pointing towards modification-specific activities. Notably and surprisingly, both Pam-2 and Pam-3 consistently inhibited S. Typhimurium growth, a species the non-modified octapeptide failed to inhibit.

### Example 2

### Screening of Octapeptide Derived Lipopeptides for Bactericidal Activity

Methods: Broth microdilution assay. Log-phase bacteria were collected by centrifugation (2500 rpm, 10 min, 4° C.), washed twice with 10 mM sodium phosphate buffer containing 1% (w/v) TSB and the optical density at 600 nm was adjusted to 0.1. Approximately 5×10⁵ CFU/ml bacteria were incubated with serial peptide concentrations (1.17-150 μM) in a final volume of 100 μl in 10 mM sodium phosphate buffer containing 1% (w/v) TSB for 2 hours at 37° C. After incubation, 100 μl of 6% TSB (w/v) was added and absorbance was measured at 600 nm (Tecan, Switzerland) and monitored for 18 hours. Afterwards, 100 μl per well was plated on LB agar to determine the number of viable bacteria microbiologically. Bactericidal activity was expressed as the LC_{99.9}, the lowest concentration that killed ≥99.9% of bacteria.

For time-kill experiments, bacteria (5×10⁵ CFU/ml) were incubated with 9.38 μM Pam-3 in 10 mM sodium phosphate buffer containing 1% (w/v) TSB in LoBind tubes (Eppendorf) in a total volume of 550 μl. As an untreated control, bacteria were incubated in 10 mM sodium phosphate buffer containing 1% (w/v) TSB. After incubation at 37° C. and 150 rpm for 1 to 120 min, a sample of 50 μl was taken from the suspension and added to 50 μl of a 0.05% (v/v) sodium polyanethol sulfonate (Sigma-Aldrich) solution, which neutralizes remaining peptide activity, and plated on LB agar to determine the number of viable bacteria.

Results: Pam-1 and Pam-4 displayed no or low bactericidal activity (FIG. 25), whereas Pam-5 surprisingly and contrary to the radial diffusion assay showed moderate effects against these pathogens. Similar to the results of the radial diffusion assay, both Pam-2 and Pam-3 were highly effective. Remarkably, Pam-2 and Pam-3 surprisingly inhibited the growth of an A. baumannii isolate (DSM30007), which is otherwise resistant to the last-resort antibiotics, colistin and tigecycline (FIG. 3). Despite some bactericidal similarities between Pam-2 and Pam-3, the latter proved superior to all other Pam's and highly effective against these bacteria at concentrations of 4.69 to 18.75 μM (FIG. 25).

### Example 3

### Screening of Octapeptide Based Lipopeptides for Antifungal Activity

Methods: Broth microdilution assay. Candida was cultured in TSB over night at 37° C., centrifuged (1500 rpm, 10 min, RT) and washed twice with 10 mM sodium phosphate buffer containing 1% (w/v) TSB. Candida cells were counted and approximately 5×10⁵ CFU/ml fungi were incubated with serial peptide concentrations (1.17-150 μM) in a final volume of 100 μl in 10 mM sodium phosphate buffer containing 1% (w/v) TSB for 2 hours at 37° C. After incubation, 100 μl of 6% TSB (w/v) was added and absorbance was measured at 600 nm (Tecan, Switzerland) and monitored for 18 hours. Afterwards, 100 μl per well was plated on YPD agar to determine the number of viable bacteria microbiologically.

Results: Pam-1 and Pam-4 displayed no antifungal activity against neither C. albicans nor against C. tropicalis. Pam-2 showed modest antifungal activity against C. tropicalis but surprisingly no activity against C. albicans. Pam-5 surprisingly showed good activity against C. tropicalis and modest activity against C. albicans. Pam-3 demonstrated very strong activity against both C. tropicalis and C. albicans (FIG. 4).

### Example 4

### Assessment of Biofilm Eradication.

Methods: Biofilm degradation assay. A log-phase culture of P. aeruginosa was diluted in BM2 medium and of S. aureus in TSB to 5×10⁵ CFU/ml. 100 μl of each bacterial suspension was added to a round-bottom polystyrene microtiter plate and incubated for 24 hours at 37° C. in a humidified atmosphere. Then, planktonic bacteria were removed by two wash steps with PBS. Next, biofilms were exposed to serial peptide dilutions (9.38-300 μM) in a final volume of 100 μl in 10 mM sodium phosphate buffer containing 1% (w/v) TSB for 1 hour at 37° C. in a humidified atmosphere. As a control, bacteria were exposed to 10 mM sodium phosphate buffer containing 1% (w/v) TSB without peptide. Afterwards, adherent bacteria in each well were resuspended, and the number of viable bacteria was determined microbiologically. To visualize the data on a logarithmic scale, a value of 1 CFU was assigned when no growth occurred.

Results: Bacterial biofilms are highly resistant to growth inhibitors and bactericidal treatment regimens. Apart from the hindered penetration of antibacterial agents, treatment is further complicated by 10 to 1000 times increased tolerance exhibited by biofilm protected bacteria compared to planktonic bacteria. Because of that, we assessed the ability of Pam-3 to eradicate established biofilms in a dose-depended manner. Within 1 hour, 300 μM of Pam-3 eliminated P. aeruginosa biofilms and similarly eradicated -99.99% of S. aureus in biofilms (FIG. 5).

### Example 5

### Assessment of Development of Bacterial Resistance.

Method: Development of resistance to the lipopeptides was assessed with S. aureusand S. Typhimurium. For comparison, the development of resistance to the clinically relevant antibiotics rifampicin and ciprofloxacin (Sigma-Aldrich) was determined. Bacteria were cultured overnight at 37° C. at 150 rpm in TSB. Bacteria were washed twice with 10 mM sodium phosphate buffer containing 1% (w/v) TSB. Washed bacteria were incubated with serial Pam-3 or antibiotic concentrations (with final concentrations of 1.17 to 150 μM peptide or 0.0156 to 0.5 μg/ml rifampicin or ciprofloxacin) in a final volume of 100 μl in 10 mM sodium phosphate buffer containing 1% (w/v) TSB for 2 hours at 37° C. After incubation, 100 μl of 6% TSB (w/v) was added and plates incubated in a humidified atmosphere for 21 hours at 37° C. and 150 rpm.

The MIC, the lowest concentration of lipopeptide/antibiotic that caused a lack of visible bacterial growth, was determined for each bacterial species. Thereafter, 5×10⁵ CFU/ml of the 0.5-fold MIC suspension was added to a fresh medium containing lipopeptides/antibiotics and these mixtures were incubated as described above. This procedure was repeated for 25 passages.

Results: The ability of S. aureus and S. Typhimurium to develop resistance against Pam-3 was assessed. When cultured in the presence of sub-inhibitory concentrations of Pam-3 for 25 passages, no significant increase in the minimal inhibition concentration (MIC) was observed for S. aureus. In contrast, the MIC for the standard antibiotic, rifampicin started to rapidly increase after five passages and had increased 4096-fold after 15 passages (FIG. 6). Similarly, although exposed to Pam-3 for continuous serial passages, no resistant S. Typhimurium isolates emerged, whereas the presence of ciprofloxacin, resulted in an increased MIC already after 3 passages, and a ≥256-fold MIC increase after 19 passages (FIG. 6).

### Example 6

### Determination of Mode of Action of Octapeptide Derived Lipopeptides.

Methods I: Interaction with the Bacterial Membrane was Determined by Reporter Gene Assay.

A specific bacterial reporter strain with the genetic background of Bacillus subitilis 1S34, carrying the promoter of the ypuA gene, fused to the firefly luciferase reporter gene, was used to identify cell envelope-related damage caused by treatment with antimicrobial compounds. Bacteria were cultured to an OD₆₀₀ of 0.9 in LB broth with 5 μg/ml erythromycin at 37° C. and diluted to an OD₆₀₀ of 0.02. Serial peptide dilutions (0.15-150 μM) were prepared in a microtiter plate and incubated with the adjusted bacterial suspension at 37° C. for 1 hour. Subsequently, citrate buffer (0.1 M, pH 5) containing 2 mM luciferin (Iris Biotech, Germany) was added and luminescence was measured using a microplate reader (Tecan, Switzerland).

### Methods II: Determination of Membrane Potential

S. aureus NCTC8325 was grown to log-phase in LB+0.1% glucose, harvested and the optical density at 600 nm (OD₆₀₀) was adjusted to 0.5. Bacteria were incubated with 30 μM 3,3′-diethyloxacarbocyanine iodide (DiOC₂(3), Invitrogen™) for 15 min in the dark and treated with serial peptide concentrations for 30 min. The protonophore carbonyl cyanide m-chlorophenyl hydrazone (CCCP, Sigma Aldrich) was used as a positive control and DMSO or 0.01% acetic acid as negative control. Fluorescence was measured at an excitation wavelength of 485 nm and two emission wavelengths, 530 nm (green) and 630 (nm) red, using a microplate reader (Tecan, Switzerland).

### Methods III: Determination of Bacterial Pore Formation

Pore formation was monitored using the Live/Dead BacLight bacterial viability kit (Molecular Probes). S. aureus was grown in LB at 37° C. to log-phase and, 100 μl aliquots were treated with 37.5 μM Pam-3 (4× MIC) or left untreated as a control. Samples were taken after 10 min of peptide treatment, then 0.2 μl of a 1:1 mixture of SYTO9 and propidium iodide (PI) was added and further incubated for 15 min at RT in the dark. Fluorescence microscopy was carried out using a Zeiss Axio Observer Z1 automated microscope. Images were acquired with an Orca Flash 4.0 V2 camera g(Hamamatsu), C Plan-Apo 63×/1.4 Oil DIC and alpha Plan-Apochromat 100×/ 1.46 Oil Ph3 objectives (Zeiss) and processed using the Zen software package (Zeiss).

### Method IV: Transmission and Scanning Electron Microscopy.

Electron microscopy for morphologic analysis of bacteria was performed.

Approximately 1.2×109 CFU/ml bacteria were incubated with 150 μM Pams or 37.5 μM Pam-3 (4× MIC) in 10 mM sodium phosphate buffer containing 1% (w/v) TSB broth for 30 or 120 min at 37° C. As a control, bacteria were exposed to 0.01% acetic acid. Afterwards bacteria were fixed in Karnovsky's reagent.

For transmission electron microscopy of FIG. 11, bacteria were high-pressure frozen (HPF Compact 03, Engineering Office M. Wohlwend GmbH) in capillaries, freeze-substituted (AFS2, Leica Microsystems) with 2% OsO₄ and 0.4% uranyl acetate in acetone as substitution medium and embedded in EPON. Ultrathin sections were stained with uranyl acetate and lead citrate and analyzed with a Tecnai Spirit (Thermo Fisher Scientific) operated at 120 kV.

For transmission electron microscopy (TEM) of FIGS. 12 & 13, post-fixed samples (1% OsO4, 1 h) were rinsed with distilled water, block-stained with uranyl acetate (2% in distilled water), dehydrated in alcohol (stepwise 30-96%), immersed in propylene oxide and embedded in glycine ether (polymerized 48 h at 60 C, Serva, Heidelberg). Ultra-thin sections were examined with a LIBRA 120 (Carl Zeiss AG, Oberkochen) at 120 kV. For scanning electron microscopy (SEM), bacteria were washed in PBS and finally fixed with 1% OsO₄ on ice for 1 hour. Next, samples were prepared on polylysin-coated coverslips, dehydrated in a graduated series to 100% ethanol and critical point dried (Polaron) with CO₂. Finally, samples were sputter-coated with a 3 nm thick layer of platinum (Safematic CCu-010) and examined with a Hitachi Regulus 8230 field emission scanning electron microscope (Hitachi) at an accelerating voltage of 5 kV.

Results: A primary target of antimicrobial peptides is the bacterial cell membrane. Disturbing the integrity and function of the outer and/or inner membranes results in loss of the barrier function and dissipation of the membrane potential. To clarify the mode of action of the octapeptide derived lipopeptides, we used a ypuA promotor-based luciferase reporter strain of B. subtilis to identify cytoplasmic membrane-associated and cell envelope-related stress. The ypuA promoter was activated (2 fold) by Pam-2 and Pam-3, indicating cell envelope impairment (FIG. 7).

The influence of the octapeptide derived lipopeptides on the transmembrane potential of S. aureus NCTC8325 was assessed. The protonophore carbonyl cyanide m-chlorophenyl hydrazone (CCCP) was used as a positive control to depolarize bacteria, i.e. leading to a reduction of their membrane potential. Upon depolarization, DiOC₂(3) shifts from green fluorescence towards red emission because of self-association of the dye molecules. Treatment with any of the lipopeptides caused a breakdown of the membrane potential in a concentration-dependent manner most pronounced for Pam-2, Pam-3 and Pam-5 (FIG. 8). Both results emphasize that the octapeptide derived lipopeptides act on bacterial membranes.

Since a large variety of AMPs target the membrane as pore formers, the ability of Pam-3 to induce membrane lesions was analyzed. S. aureus NCTC8325 was treated with Pam-3 at 4× MIC and added a mixture of Syto9 and propidium iodide (PI). The membrane-permeant Syto9 stains all living cells green, whereas the red-fluorescent PI can only enter cells through large membrane pores or lesions. Pam-3 led to a strong influx of PI (FIG. 9). Pore formation was associated with fast killing of these bacteria. We assayed bactericidal kinetics to assess the rapidness of Pam-3 mediated killing. Pam-3 killed more than 90% of P. aeruginosa within 1 min and S. aureus within 15 min (FIG. 10). Eradication to the level of detection was observed 2 and 30 min after Pam-3 treatment, respectively.

Scanning electron microscopy (SEM) was employed to observe cell morphological changes after Pam-3 treatment directly. Exposure to Pam-3 resulted in membrane surface disruption and lysed cells similar to hBD1 (Raschig et al., 2017), while control cells exhibited a bright and smooth surface (FIG. 11).

Transmission electron microscopy (TEM) was performed to analyze changes in bacterial morphology to compare bacterial morphology before and after treatment with the lipopeptides in S. aureus and S. Enteritidis. In agreement with the pore formation and the induction of cell envelope stress and depolarization, treatment with octapeptide derived lipopeptides resulted in strong cell envelope damage with disrupted membranes and pores in most of the cells (FIGS. 12 and 13).

### Example 7

Identification of Possible Cell Wall Targets in E. coli and S. aureus Mutants.

E. coli BW 25113 mutants differ in their LPS composition which makes them useful to study the binding of Pams to Gram negative bacteria. For comparison, E. coli BW 25113 and E. coli ATCC 25922 were utilized as control. E. coli ATCC 25922 possess the full-length LPS thus additional an O-antigen while E. coli BW 25113 lacks the O-antigen. The E. coli BW 25113 mutant ΔwaaG has similar phosphate residues like the wild type thus the same charge but is lacking an outer core. The mutant ΔwaaY possess an outer core while lacking one phosphate residue in the inner core leading to a less negative charge of the cell wall. A similar LPS composition has the mutant ΔwaaP except the amount of phosphate residues in the inner core which is zero. In order to identify cell wall targets of Gram-positive bacteria, cell wall mutants of S. aureus SA113 were investigated regarding their susceptibility against the Pams. S. aureus SA113 was used as control. The ΔdItA mutant lacks D-alanine in the peptidoglycan layer resulting in a more negative charge which should facilitate the binding of Pams and subsequent cell wall disruption (VVeidenmaier et al., 2005). A similar cell wall composition has the ΔmprF mutant lacking L-lysin in the cell membrane resulting in a more negative charge (Peschel and Collins, 2001). Additional teichoic acid in the peptidoglycan layer possess the tarH mutant causing a strengthening of it (Wanner et al., 2017).

Methods: Broth microdilution assay. Log-phase bacteria were collected by centrifugation (2500 rpm, 10 min, 4° C.), washed twice with 10 mM sodium phosphate buffer containing 1% (w/v) TSB and the optical density at 600 nm was adjusted to 0.1. Approximately 5×10⁵ CFU/ml bacteria were incubated with serial peptide concentrations (1.17-150 μM) in a final volume of 100 μl in 10 mM sodium phosphate buffer containing 1% (w/v) TSB for 2 hours at 37° C. After incubation, 100 μl of 6% TSB (w/v) was added and absorbance was measured at 600 nm (Tecan, Switzerland) and monitored for 18 hours. Afterwards, 100 μl per well was plated on LB agar to determine the number of viable bacteria microbiologically.

Results: The antimicrobial activity of Pams is independent of LPS and bacterial cell wall charge (FIGS. 14 and 15).

### Example 8

### Assessment of Tolerability of Octapeptide Derived Lipopeptides.

Methods: Cellular toxicity was determined by Lactate dehydrogenase concentration and WST-1 cell proliferation assay as well as hemolysis assay.

Results: Pam-1, Pam-4 and Pam-5 displayed no LDH activity. Pam-3 showed little LDH activity and Pam-2 showed some LDH activity (FIG. 16). Pam-1, Pam-4 and Pam-5 displayed normal metabolic activity, Pam-3 a slight decrease and Pam-2 a modest decrease of metabolic activity (FIG. 17). Pam-1, Pam-2, Pam-4 and Pam-5 showed minimal red blood cell disruption whereas Pam-3 showed modest red blood cell disruption (FIG. 18).

The Pams are thus surprisingly non-toxic considering their high efficacy.

### Example 9

Assessment of Acute Tolerability after Oral Administration.

Methods: C57BL/6N mice were generated and maintained (including breeding and housing) at the animal facilities of the Helmholtz Centre for Infection Research (HZI) under enhanced specific pathogen-free (SPF) conditions (Stehr et al., 2009). Animals used in the experiments were gender and age matched. Female and male mice with an age of 8-12 weeks were used. Sterilized food and water ad libitum were provided. Mice were kept under a strict 12-hour light cycle (lights on at 7:00 am and off at 7:00 μm) and housed in groups of two to six mice per cage. All mice were euthanized by asphyxiation with CO₂ and cervical dislocation. All animal experiments were performed in agreement with the local government of Lower Saxony, Germany (approved permission No. 33.19-42502-04-18/2499).

Age- and gender-matched mice received two doses of peptides (0, 125 μg or 250 μg) solved in 100 μl PBS orally per day. Bodyweight and appearance were recorded. The following day, mice were sacrificed and stomach, kidney, spleen, liver, small intestine, cecum and colon were removed for histological scoring. Around 1 ml of blood was taken from the heart to measure inflammatory markers including creatinine in the kidney and the enzyme levels of glutamate-oxalacetate-transaminase (GOT) in the liver.

Results: Histological analysis and determination of serum markers 24 hours after application of two doses of 250 μg of Pam-1 or Pam-3 did not reveal any acute toxicity. Specifically, there were no alterations in bodyweight and no signs of systemic toxicity or distress (FIG. 19). Moreover, measurement of serum levels of creatinine and GOT showed no significant differences between the groups suggesting no effect on kidney and liver metabolism (FIG. 19). Finally, histological examination of gastrointestinal, liver and kidney tissues revealed no alterations, except a minor shortening of intestinal villi the jejunum-ileum of one control and two treated animals (FIG. 19). As no difference in these histopathological findings was observed between PBS and Pam-3 treated animals, they were regarded as background observations (FIG. 26). Thus, we conclude that Pam-1 and Pam-3 treatment was not associated with acute toxicity.

### Example 10

Treatment of Acute Gastrointestinal Infection Caused by S. typhimurium.

Methods: Analysis of bacterial loads in feces. Fresh fecal samples were collected and weighted. Samples were homogenized in 1 ml LB media by bead-beating with 1 mm zirconia/silica beads twice for 25 s using a Mini-Beadbeater-96 (BioSpec). To determine CFUs, dilutions of homogenized samples were plated on LB plates with 50 μg/ml kanamycin.

Analysis of bacterial loads in intestinal content and systemic organs.

All mice were euthanized by asphyxiation with CO₂ at indicated time points. Intestinal tissues (small intestine, cecum, colon) were removed aseptically. To collect intestinal content, organs were flushed with PBS. Organs were opened longitudinally, cleaned thoroughly with PBS and weighted. Organs and content were homogenized in PBS using a Polytron homogenizer (Kinemtatica). Dilutions of homogenized samples were plated on LB plates containing 50 μg/ml kanamycin to determine CFUs.

For S. Typhimurium infection experiments, age- and sex-matched mice between 10 and 14 weeks of age were used. Both—female and male mice were used in experiments. Water and food were withdrawn for 4 hours before mice were treated with 20 mg/mouse of streptomycin by oral gavage. Afterwards, mice were supplied with water and food ad libitum. 20 hours after streptomycin treatment, water and food were withdrawn again, 4 hours before the mice were orally infected with 10⁵ CFU of S. Typhimurium in 200 μl PBS. Drinking water ad libitum was supplied immediately and food 2 hours post infection (p.i.). After 6 and 22 hours p.i. mice received 250 μg lipopeptide solved in 100 μl PBS or only PBS orally. 48 hours after infection, mice were sacrificed, and intestinal organs were removed to assess the bacterial burden in the lumen and tissues. Mice were weighed every day to record potential body-weight loss. Mice were infected with S. Typhimurium and treated orally 6 and 22 hours post infection with 250 μg Pam-1 or Pam-3 or PBS.

Results: Pam-1 treated animals showed significantly reduced Colony Forming Units (CFU) of S. Typhimurium in cecum content but not in tissue (Mann-Whitney test, p<0.017, FIG. 20).

Pam-3 treated animals tend to show reduced weight loss (FIG. 21) and showed significantly reduced CFU's of S. Typhimurium in cecum content and tissue (Mann-Whitney test, p<0.0001 and 0.05, FIG. 21). Furthermore, Pam-3 also lowered the bacterial load in the small intestine without affecting the small intestine tissue (Mann-Whitney test, p<0.001, FIG. 21). This example demonstrates the efficacy of the Pams and Pam-3 in particular in eradicating acute gastrointestinal infection.

### Example 11

Treatment of an Established Gastrointestinal Infection Caused by C. rodentium.

Methods: Citrobacter rodentium infection. Bioluminescence expressing C. rodentium strain ICC180 was used for all infection experiments (Wiles et al., 2004). C. rodentium inoculae were prepared by culturing bacteria overnight at 37° C. in LB broth with 50 μg/mI kanamycin. Subsequently, the culture was diluted 1:100 in fresh medium, and sub-cultured for 4 hours at 37° C. in LB broth (Thiemann et al., 2017). Bacteria were washed twice in phosphate-buffered saline (PBS). Mice were orally inoculated with 10⁸ CFU of C. rodentium diluted in 200 μl PBS. Weight of the mice was monitored and feces was collected for measurements of the pathogen burden. 5 days post infection mice received twice 250 μg peptide solved in 100 μl PBS or only PBS orally. The following day, mice were sacrificed to assess bacterial burden in the lumen and tissues of the cecum and the colon.

Mice were infected with C. rodentium and received two doses of 250 μg Pam-1, Pam-3 or PBS 5 days post infection.

Results: Pam-1 treatment surprisingly did not reduce CFU's in cecum (FIG. 20). Pam-3 treatment reduced the number of bacteria in the cecum content and cecum tissue (Mann-Whitney test, p<0.05 and 0.05, FIG. 21). Treatment also significantly reduced the number of viable Citrobacter in colon content and colon tissue (Mann-Whitney test, p<0.01 and p<0.05). Together these data corroborate the in vivo efficacy of Pams and Pam-3 in particular against two different enteric pathogens.

### Example 12

### Microbiome Analysis Following Administration of Pams.

Methods: Mice were treated twice at an 8-hour interval with Pam-1 or Pam-3 (125 or 250 μg/each dose) or PBS orally and fresh fecal samples were collected before and 24 hours after application. Microbiota composition was analyzed using 16S rRNA sequencing. Feces samples were collected at different time points (before and after infection), and bacterial DNA was extracted from snap-frozen feces using a phenol-chloroform-based method previously described. In brief, 500 μl of extraction buffer (200 mM Tris (Roth), 20 mM EDTA (Roth), 200 mM NaCl (Roth), pH 8.0), 200 μl of 20% SDS (AppliChem), 500 μl of phenol:chloroform:isoamyl alcohol (PCI) (24:24:1) (Roth) and 100 μl of zirconia/silica beads (0.1 mm diameter) (Roth) were added per feces sample. Lysis of bacteria was performed by mechanical disruption using a Mini-BeadBeater-96 (BioSpec) for two times 2 min. After centrifugation, the aqueous phase was processed by another phenol:chloroform:isoamyl alcohol extraction before precipitation of DNA using 500 μl isopropanol (J. T. Baker) and 0.1 volume of 3 M sodium acetate (Applichem). Samples were incubated at −20° C. for at least several hours or overnight and centrifuged at 4° C. at maximum speed for 20 min. The resulting DNA pellet was washed, dried using a speed vacuum and resuspended in TE Buffer (Applichem) with 100 μg/ml RNase I (Applichem). Crude DNA was column purified (BioBasic Inc.) to remove PCR inhibitors.

16S rRNA gene amplification of the V4 region (F515/R806) was performed according to an established protocol. Briefly, DNA was normalized to 25 ng/μl and used for sequencing PCR with unique 12-base Golary barcodes incorporated via specific primers (obtained from Sigma). PCR was performed using Q5 polymerase (NewEnglandBiolabs) in triplicates for each sample, using PCR conditions of initial denaturation for 30 s at 98° C., followed by 25 cycles (10 s at 98° C., 20 s at 55° C., and 20 s at 72° C.). After pooling and normalization to 10 nM, PCR amplicons were sequenced on an Illumina MiSeq platform via 250 bp paired-end sequencing (PE250). Using Usearch8.1 software package (http://www.drive5.com/usearch/) the resulting reads were assembled, filtered and clustered. Sequences were filtered for low-quality reads and binned based on sample-specific barcodes using QIIME v1.8.0 (Caporaso et al., 2010). Merging was performed using—fastq_mergepairs—with fastq maxdiffs 30. Quality filtering was conducted with fastq_filter (-fastq_maxee 1), using a minimum read length of 250 bp and a minimum number of reads per sample=1000. Reads were clustered into 97% ID OTUs by open-reference OTU picking and representative sequences were determined by use of UPARSE algorithm (Edgar, 2010). Abundance filtering (OTUs cluster >0.5%) and taxonomic classification were performed using the RDP Classifier executed at 80% bootstrap confidence cut off. Sequences without matching reference dataset were assembled as de novo using UCLUST. Phylogenetic relationships between OTUs were determined using FastTree to the PyNAST alignment. Resulting OTU absolute abundance table and mapping file were used for statistical analyses and data visualization in the R statistical programming environment package phyloseq.

Results: Analysis of beta diversity and calculation of weighted Unifrac Distances demonstrated that changes in the microbiota between the before and after samples were similar between Pam-1 and Pam-3 treated mice and PBS gavaged control mice (FIGS. 22 & 23). Similarly, while minor changes in the community structure were observed in both groups (i.e. treated and untreated), the number of detected species as well as the complexity (FIG. 22) surprisingly remained comparable, thus contrasting treatment with traditional antibiotics, such as ampicillin (FIG. 24). In line with the analysis of alpha and beta diversity, Pam-1 or Pam-3 treatment surprisingly did not affect the abundance of bacterial genera (FIGS. 22 & 23). Combined, these results demonstrate that, with the application regime conducted, Pam-1 or Pam-3 treatment of healthy chow-fed mice surprisingly does not affect the overall community structure or diversity of the microbiota.

### Example 13

Microbiome analysis following administration of Pam-3, Ampicillin and Levofloxacin Methods: Chow-fed mice were treated twice at an 8-hour interval with Pam-3 (125 or 250 μg/each dose) or once with Levofloxacin (2 mg solved in 100 μl PBS) or Ampicillin (5 g/L in drinking water) or PBS orally and collected fresh fecal samples before and 24 hours after application.

Microbiota composition was analyzed using 16S rRNA sequencing. Feces samples were collected at different time points (before and after infection), and bacterial DNA was extracted from snap-frozen feces using a phenol-chloroform-based method previously described. In brief, 500 μl of extraction buffer (200 mM Tris (Roth), 20 mM EDTA (Roth), 200 mM NaCl (Roth), pH 8.0), 200 μl of 20% SDS (AppliChem), 500 μl of phenol:chloroform:isoamyl alcohol (PCI) (24:24:1) (Roth) and 100 μl of zirconia/silica beads (0.1 mm diameter) (Roth) were added per feces sample. Lysis of bacteria was performed by mechanical disruption using a Mini-BeadBeater-96 (BioSpec) for two times 2 min. After centrifugation, the aqueous phase was processed by another phenol:chloroform:isoamyl alcohol extraction before precipitation of DNA using 500 μl isopropanol (J. T. Baker) and 0.1 volume of 3 M sodium acetate (Applichem). Samples were incubated at −20° C. for at least several hours or overnight and centrifuged at 4° C. at maximum speed for 20 min. The resulting DNA pellet was washed, dried using a speed vacuum and resuspended in TE Buffer (Applichem) with 100 μg/ml RNase I (Applichem). Crude DNA was column purified (BioBasic Inc.) to remove PCR inhibitors. 16S rRNA gene amplification of the V4 region (F515/R806) was performed according to an established protocol. Briefly, DNA was normalized to 25 ng/μl and used for sequencing PCR with unique 12-base Golary barcodes incorporated via specific primers (obtained from Sigma). PCR was performed using Q5 polymerase (NewEnglandBiolabs) in triplicates for each sample, using PCR conditions of initial denaturation for 30 s at 98° C., followed by 25 cycles (10 s at 98° C., 20 s at 55° C., and 20 s at 72° C.). After pooling and normalization to 10 nM, PCR amplicons were sequenced on an Illumina MiSeq platform via 250 bp paired-end sequencing (PE250). Using Usearch8.1 software package (http://www.drive5.com/usearch/) the resulting reads were assembled, filtered and clustered. Sequences were filtered for low-quality reads and binned based on sample-specific barcodes using QIIME v1.8.0. Merging was performed using -fastq_mergepairs—with fastq_maxdiffs 30. Quality filtering was conducted with fastq_filter (-fastq_maxee 1), using a minimum read length of 250 bp and a minimum number of reads per sample=1000. Reads were clustered into 97% ID OTUs by open-reference OTU picking and representative sequences were determined by use of UPARSE algorithm. Abundance filtering (OTUs cluster >0.5%) and taxonomic classification were performed using the RDP Classifier executed at 80% bootstrap confidence cut off. Sequences without matching reference dataset were assembled as de novo using UCLUST. Phylogenetic relationships between OTUs were determined using FastTree to the PyNAST alignment. Resulting OTU absolute abundance table and mapping file were used for statistical analyses and data visualization in the R statistical programming environment package phyloseq.

Results: Analysis of observed species, bacterial diversity and abundance of bacterial genera showed significant changes in the microbiota after Lenofloxacin or Ampicillin treatment. In contrast and surprisingly, the administration of Pam-3 caused no or only slight differences. These results demonstrate the major advantages of Pam-3 over common antibiotics.

### Example 14

Tables with the sequences of hBD-1, HDS, HD6 and HNP4 fragments that can be generated in silico versus the defensin fragments that can be identified biologically.

| In silico digestion of hBD1 with | | | | |
|---|---|---|---|---|
| intestinal proteases | | | Fragments which can be | |
| hBD1 | | | identified with mass | |
| | missed | | spectometry | SEQ ID |
| position | cleavages | sequence | sequence | NO: |
| 1-36 | 5 | [symbol] | [symbol] | 1 |
| | | [symbol] | [symbol] | |
| | | [symbol] | [symbol] | |
| 1-33 | 4 | DHYNCVSSGGQCLYSAC | | 2 |
| | | PIFTKIQGTCYRGKAK | | |
| 4-36 | 4 | NCVSSGGQCLYSACPIFT | | 3 |
| | | KIQGTCYRGKAKCCK | | |
| 1-31 | 3 | DHYNCVSSGGQCLYSAC | | 4 |
| | | PIFTKIQGTCYRGK | | |
| 1-29 | 1 | DHYNCVSSGGQCLYSAC | | 5 |
| | | PIFTKIQGTCYR | | |
| 1-28 | 4 | DHYNCVSSGGQCLYSAC | DHYNCVSSGGQC | 6 |
| | | PIFTKIQGTCY | LYSACPIFTKIQGT | |
| | | | CY | |
| 4-31 | 5 | NCVSSGGQCLYSACPIFT | | 7 |
| | | KIQGTCYRGK | | |
| 4-29 | | NCVSSGGQCLYSACPIFT | | 8 |
| | | KIQGTCYR | | |
| 4-28 | 3 | NCVSSGGQCLYSACPIFT | | 9 |
| | | KIQGTCY | | |
| 14-36 | 3 | YSACPIFTKIQGTCYRGKA | | 10 |
| | | KCCK | | |
| 1-22 | 0 | DHYNCVSSGGQCLYSAC | | 11 |
| | | PIFTK | | |
| 15-36 | 2 | SACPIFTKIQGTCYRGKAK | | 12 |
| | | CCK | | |
| 1-20 | 3 | DHYNCVSSGGQCLYSAC | | 13 |
| | | PIF | | |
| 15-33 | 5 | SACPIFTKIQGTCYRGKAK | | 14 |
| 4-22 | 2 | NCVSSGGQCLYSACPIFT | | 15 |
| | | K | | |
| 15-31 | 4 | SACPIFTKIQGTCYRGK | | 16 |
| 21-36 | 5 | TKIQGTCYRGKAKCCK | | 17 |
| 4-20 | 2 | NCVSSGGQCLYSACPIF | | 18 |
| 21-36 | 1 | TKIQGTCYRGKAKCCK | | 19 |
| 15-29 | 3 | SACPIFTKIQGTCYR | | 20 |
| 14-28 | 2 | YSACPIFTKIQGTCY | | 21 |
| 23-36 | | IQGTCYRGKAKCCK | | 22 |
| 1-14 | 2 | DHYNCVSSGGQCLY | | 23 |
| 23-36 | 3 | IQGTCYRGKAKCCK | | 24 |
| 15-28 | 1 | SACPIFTKIQGTCY | | 25 |
| 1-13 | 1 | DHYNCVSSGGQCL | | 26 |
| 4-14 | 1 | NCVSSGGQCLY | | 27 |
| 21-33 | 4 | TKIQGTCYRGKAK | | 28 |
| 21-31 | 3 | TKIQGTCYRGK | | 29 |
| 23-33 | 2 | IQGTCYRGKAK | | 30 |
| 4-14 | 0 | NCVSSGGQCLY | | 31 |
| 4-13 | 0 | NCVSSGGQCL | | 32 |
| 21-29 | 2 | TKIQGTCYR | | 33 |
| 23-31 | 1 | IQGTCYRGK | | 34 |
| 21-28 | 0 | TKIQGTCY | | 35 |
| 29-36 | 0 | RGKAKCCK | RGKAKCCK | 36 |
| 15-22 | | SACPIFTK | | 37 |
| 23-29 | 0 | IQGTCYR | | 38 |
| 30-36 | 2 | GKAKCCK | GKAKCCK | 39 |
| 23-28 | 0 | IQGTCY | | 40 |
| 14-20 | 1 | YSACPIF | | 41 |
| 15-20 | 0 | SACPIF | | 42 |
| 29-33 | 2 | RGKAK | | 43 |
| 32-36 | 1 | AKCCK | | 44 |

In silico (without color) digestion of hBD1 with trypsin or chymotrypsin or both in combination, maximum of 5 missed cleavages and fragments bigger than 500 Da identified as their monoisotopic mass as [M + H]+ using the ExPASy PeptideMass module. Fragments which can be identified with mass spectrometry are bold and underlined. Underlined, bold and italic is the full-length peptide.

| In silico digestion of HD5 with | | | | |
|---|---|---|---|---|
| intestinal proteases | | | Fragments which can be | |
| HD5 | | | identified with mass | |
| | missed | | spectometry | SEQ ID |
| position | cleavages | sequence | sequence | NO: |
| 1-32 | 5 | [symbol] | [symbol] | 89 |
| | | [symbol] | [symbol] | |
| | | | [symbol] | |
| 1-29 | 4 | ATCYCRTGRCATRESLS | | 90 |
| | | GVCEISGRLYRL | | |
| 5-32 | 4 | CRTGRCATRESLSGVCEI | | 91 |
| | | SGRLYRLCCR | | |
| 1-28 | 4 | ATCYCRTGRCATRESLS | ATCYCRTGRCAT | 92 |
| | | GVCEISGRLYR | RESLSGVCEISGR | |
| | | | LYR | |
| 1-27 | 3 | ATCYCRTGRCATRESLS | ATCYCRTGRCAT | 93 |
| | | GVCEISGRLY | RESLSGVCEISGR | |
| | | | LY | |
| 7-32 | 4 | TGRCATRESLSGVCEISG | TGRCATRESLSGV | 94 |
| | | RLYRLCCR | CEISGRLYRLCCR | |
| 1-26 | 2 | ATCYCRTGRCATRESLS | | 95 |
| | | GVCEISGRL | | |
| 5-29 | 3 | CRTGRCATRESLSGVCEI | | 96 |
| | | SGRLYRL | | |
| 1-25 | 3 | ATCYCRTGRCATRESLS | ATCYCRTGRCAT | 97 |
| | | GVCEISGR | RESLSGVCEISGR | |
| 5-28 | 5 | CRTGRCATRESLSGVCEI | | 98 |
| | | SGRLYR | | |
| 10-32 | 3 | CATRESLSGVCEISGRLY | CATRESLSGVCEI | 99 |
| | | RLCCR | SGRLYRLCCR | |
| 5-27 | 2 | CRTGRCATRESLSGVCEI | | 100 |
| | | SGRLY | | |
| 7-28 | 3 | TGRCATRESLSGVCEISG | | 101 |
| | | RLYR | | |
| 5-26 | 1 | CRTGRCATRESLSGVCEI | | 102 |
| | | SGRL | | |
| 7-27 | 3 | TGRCATRESLSGVCEISG | TGRCATRESLSGV | 103 |
| | | RLY | CEISGRLY | |
| 5-25 | 3 | CRTGRCATRESLSGVCEI | | 104 |
| | | SGR | | |
| 14-32 | 2 | ESLSGVCEISGRLYRLCC | ESLSGVCEISGRL | 105 |
| | | R | YRLCCR | |
| 10-28 | 2 | CATRESLSGVCEISGRLY | CATRESLSGVCEI | 106 |
| | | R | SGRLYR | |
| 7-25 | 2 | TGRCATRESLSGVCEISG | | 107 |
| | | R | | |
| 10-27 | 2 | CATRESLSGVCEISGRLY | | 108 |
| 17-32 | 3 | SGVCEISGRLYRLCCR | | 109 |
| 1-16 | 1 | ATCYCRTGRCATRESL | | 110 |
| 14-28 | 1 | ESLSGVCEISGRLYR | | 111 |
| 10-25 | 1 | CATRESLSGVCEISGR | | 112 |
| 14-27 | 1 | ESLSGVCEISGRLY | | 113 |
| 1-13 | 2 | ATCYCRTGRCATR | ATCYCRTGRCAT | 114 |
| | | | R | |
| 17-29 | 2 | SGVCEISGRLYRL | | 115 |
| 5-16 | 0 | CRTGRCATRESL | | 116 |
| 14-25 | 0 | ESLSGVCEISGR | | 117 |
| 17-27 | 1 | SGVCEISGRLY | | 118 |
| 1-9 | 1 | ATCYCRTGR | ATCYCRTGR | 119 |
| 5-13 | 2 | CRTGRCATR | | 120 |
| 17-26 | 0 | SGVCEISGRL | | 121 |
| 26-32 | 1 | LYRLCCR | LYRLCCR | 122 |
| 27-32 | 2 | YRLCCR | | 123 |
| 7-13 | 1 | TGRCATR | | 124 |
| 1-6 | 0 | ATCYCR | | 125 |
| 28-32 | 1 | RLCCR | | 126 |
| 5-9 | 1 | CRTGR | | 127 |

In silico (without color) digestion of HD5 with trypsin or chymotrypsin or both in combination, maximum of 5 missed cleavages and fragments bigger than 500 Da identified as their monoisotopic mass as [M + H]+ using the ExPASy PeptideMass module. Fragments which can be identified with mass spectrometry are bold and underlined. Underlined, bold and italic is the full-length peptide.

| In silico digestion of HD6 with | | | | |
|---|---|---|---|---|
| intestinal proteases | | | Fragments which can be | |
| HD6 | | | identified with mass | |
| | missed | | spectometry | SEQ ID |
| position | cleavages | sequence | sequence | NO: |
| 1-32 | 3 | [symbol] | [symbol] | 128 |
| | | [symbol] | [symbol] | |
| | | | [symbol] | |
| 3-32 | 5 | TCHCRRSCYSTEYSYGT | | 129 |
| | | CTVMGINHRFCCL | | |
| 1-29 | 5 | AFTCHCRRSCYSTEYSY | | 130 |
| | | GTCTVMGINHRF | | |
| 1-28 | 2 | AFTCHCRRSCYSTEYSY | | 131 |
| | | GTCTVMGINHR | | |
| 3-29 | 4 | TCHCRRSCYSTEYSYGT | | 132 |
| | | CTVMGINHRF | | |
| 3-28 | 5 | TCHCRRSCYSTEYSYGT | | 133 |
| | | CTVMGINHR | | |
| 8-32 | 2 | RSCYSTEYSYGTCTVMGI | | 134 |
| | | NHRFCCL | | |
| 9-32 | 1 | SCYSTEYSYGTCTVMGIN | | 135 |
| | | HRFCCL | | |
| 1-23 | 4 | AFTCHCRRSCYSTEYSY | | 136 |
| | | GTCTVM | | |
| 8-29 | 5 | RSCYSTEYSYGTCTVMGI | | 137 |
| | | NHRF | | |
| 3-23 | 3 | TCHCRRSCYSTEYSYGT | | 138 |
| | | CTVM | | |
| 8-28 | 1 | RSCYSTEYSYGTCTVMGI | | 139 |
| | | NHR | | |
| 9-29 | 4 | SCYSTEYSYGTCTVMGIN | | 140 |
| | | HRF | | |
| 12-32 | 4 | STEYSYGTCTVMGINHRF | | 141 |
| | | CCL | | |
| 9-28 | 0 | SCYSTEYSYGTCTVMGIN | | 142 |
| | | HR | | |
| 1-17 | 3 | AFTCHCRRSCYSTEYSY | | 143 |
| 12-29 | 3 | STEYSYGTCTVMGINHRF | | 144 |
| 12-28 | 2 | STEYSYGTCTVMGINHR | | 145 |
| 16-32 | 3 | SYGTCTVMGINHRFCCL | | 146 |
| 3-17 | 2 | TCHCRRSCYSTEYSY | | 147 |
| 1-15 | 2 | AFTCHCRRSCYSTEY | | 148 |
| 18-32 | 2 | GTCTVMGINHRFCCL | | 149 |
| 3-15 | 1 | TCHCRRSCYSTEY | | 150 |
| 16-29 | 2 | SYGTCTVMGINHRF | | 151 |
| 16-28 | 1 | SYGTCTVMGINHR | | 152 |
| 1-11 | 1 | AFTCHCRRSCY | | 153 |
| 12-23 | 2 | STEYSYGTCTVM | | 154 |
| 18-29 | 1 | GTCTVMGINHRF | | 155 |
| 8-17 | 3 | RSCYSTEYSY | | 156 |
| 18-28 | 0 | GTCTVMGINHR | | 157 |
| 3-11 | 2 | TCHCRRSCY | | 158 |
| 9-17 | 2 | SCYSTEYSY | | 159 |
| 24-32 | 1 | GINHRFCCL | | 160 |
| 8-15 | 2 | RSCYSTEY | | 161 |
| 1-8 | 2 | AFTCHCRR | | 162 |
| 16-23 | 1 | SYGTCTVM | | 163 |
| 9-15 | 1 | SCYSTEY | | 164 |
| 1-7 | 0 | AFTCHCR | | 165 |
| 3-8 | 1 | TCHCRR | | 166 |
| 12-17 | 1 | STEYSY | | 167 |
| 24-29 | 0 | GINHRF | | 168 |
| 3-7 | 0 | TCHCR | | 169 |
| 18-23 | 0 | GTCTVM | | 170 |
| 8-11 | 1 | RSCY | | 171 |

In silico (without color) digestion of HD6 with trypsin or chymotrypsin or both in combination, maximum of 5 missed cleavages and fragments bigger than 500 Da identified as their monoisotopic mass as [M + H]+ using the ExPASy PeptideMass module. Fragments which can be identified with mass spectrometry are bold and underlined. Underlined, bold and italic is the full-length peptide.

| In silico digestion of HNP4 with | | | | |
|---|---|---|---|---|
| intestinal proteases | | | Fragments which can be | |
| HNP4 | | | identified with mass | |
| | missed | | spectrometry | SEQ ID |
| position | cleavages | sequence | sequence | NO: |
| 1-33 | 3 | [symbol] | [symbol] | 172 |
| | | [symbol] | [symbol] | |
| | | | [symbol] | |
| 1-32 | 4 | VCSCRLVFCRRTELRVGN | | 173 |
| | | CLIGGVSFTYCCTR | | |
| 1-28 | 2 | VCSCRLVFCRRTELRVGN | | 174 |
| | | CLIGGVSFTY | | |
| 6-33 | 4 | LVFCRRTELRVGNCLIGG | | 175 |
| | | VSFTYCCTRV | | |
| 6-32 | 3 | LVFCRRTELRVGNCLIGG | | 176 |
| | | VSFTYCCTR | | |
| 1-26 | 5 | VCSCRLVFCRRTELRVGN | | 177 |
| | | CLIGGVSF | | |
| 9-33 | | CRRTELRVGNCLIGGVSF | | 178 |
| | | TYCCTRV | | |
| 9-32 | 5 | CRRTELRVGNCLIGGVSF | | 179 |
| | | TYCCTR | | |
| 6-28 | 5 | LVFCRRTELRVGNCLIGG | | 180 |
| | | VSFTY | | |
| 11-33 | 5 | RTELRVGNCLIGGVSFTY | RTELRVGNCLIGG | 181 |
| | | CCTRV | VSFTYCCTRV | |
| 11-32 | 4 | RTELRVGNCLIGGVSFTY | | 182 |
| | | CCTR | | |
| 12-33 | 4 | TELRVGNCLIGGVSFTYC | | 183 |
| | | CTRV | | |
| 6-26 | 4 | LVFCRRTELRVGNCLIGG | | 184 |
| | | VSF | | |
| 12-32 | 3 | TELRVGNCLIGGVSFTYC | | 185 |
| | | CTR | | |
| 9-28 | 4 | CRRTELRVGNCLIGGVSF | | 186 |
| | | TY | | |
| 11-28 | 3 | RTELRVGNCLIGGVSFTY | | 187 |
| 9-26 | 3 | CRRTELRVGNCLIGGVSF | | 188 |
| 16-33 | 3 | VGNCLIGGVSFTYCCTRV | VGNCLIGGVSFTY | 189 |
| | | | CCTRV | |
| 1-15 | 4 | VCSCRLVFCRRTELR | VCSCRLVFCRRTE | 190 |
| | | | LR | |
| 12-28 | 2 | TELRVGNCLIGGVSFTY | | 191 |
| 16-32 | 2 | VGNCLIGGVSFTYCCTR | VGNCLIGGVSFTY | 192 |
| | | | CCTR | |
| 11-26 | 2 | RTELRVGNCLIGGVSF | | 193 |
| 12-26 | 1 | TELRVGNCLIGGVSF | | 194 |
| 1-11 | 3 | VCSCRLVFCRR | VCSCRLVFCRR | 195 |
| 16-28 | 1 | VGNCLIGGVSFTY | | 196 |
| 6-15 | 3 | LVFCRRTELR | LVFCRRTELR | 197 |
| 1-10 | 2 | VCSCRLVFCR | VCSCRLVFCR | 198 |
| 16-26 | 0 | VGNCLIGGVSF | | 199 |
| 9-15 | 2 | CRRTELR | | 200 |
| 1-8 | 1 | VCSCRLVF | | 201 |
| 27-33 | 2 | TYCCTRV | | 202 |
| 6-11 | 2 | LVFCRR | LVFCRR | 203 |
| 27-32 | 1 | TYCCTR | | 204 |
| 11-15 | 1 | RTELR | | 205 |
| 6-10 | 1 | LVFCR | LVFCR | 206 |
| 29-33 | 1 | CCTRV | | 207 |
| 1-5 | 0 | VCSCR | | 208 |
| 12-15 | 0 | TELR | | 209 |

In silico (without color) digestion of HNP4 with trypsin or chymotrypsin or both in combination, maximum of 5 missed cleavages and fragments bigger than 500 Da identified as their monoisotopic mass as [M + H]+ using the ExPASy PeptideMass module. Fragments which can be identified with mass spectrometry are bold and underlined. Underlined, bold and italic is the full-length peptide.

### REFERENCES

Bevins, C. L. (2003). Antimicrobial peptides as effector molecules of mammalian host defense. Contrib. Microbiol. 10, 106-148.
Blaser, M. J. (2016). Antibiotic use and its consequences for the normal microbiome. Science 352, 544-545.
Bowie, J. U., and Sauer, R. T. (1989). Identifying determinants of folding and activity for a protein of unknown structure. Proc. Natl. Acad. Sci. U.S.A. 86, 2152-2156.
Burdet, C., Nguyen, T. T., Duval, X., Ferreira, S., Andremont, A., Guedj, J., Mentré, F., and DAV132-CL-1002 Study Group (2019). Impact of Antibiotic Gut Exposure on the Temporal Changes in Microbiome Diversity. Antimicrob. Agents Chemother. 63.
Caporaso, J. G., Kuczynski, J., Stombaugh, J., Bittinger, K., Bushman, F. D., Costello, E. K., Fierer, N., Peña, A. G., Goodrich, J. K., Gordon, J. I., et al. (2010). QIIME allows analysis of high-throughput community sequencing data. Nat. Methods 7, 335-336.
Cole, J. N., and Nizet, V. (2016). Bacterial Evasion of Host Antimicrobial Peptide Defenses. Microbiol. Spectr. 4.
Cunningham, B. C., and Wells, J. A. (1989). High-resolution epitope mapping of hGH-receptor interactions by alanine-scanning mutagenesis. Science 244, 1081-1085.
Derbyshire, K. M., Salvo, J. J., and Grindley, N. D. (1986). A simple and efficient procedure for saturation mutagenesis using mixed oligodeoxynucleotides. Gene 46, 145-152.
Dethlefsen, L., and Relman, D. A. (2011). Incomplete recovery and individualized responses of the human distal gut microbiota to repeated antibiotic perturbation. Proc. Natl. Acad. Sci. U.S.A. 108 Suppl 1, 4554-4561.
Dethlefsen, L., Huse, S., Sogin, M. L., and Relman, D. A. (2008). The pervasive effects of an antibiotic on the human gut microbiota, as revealed by deep 16S rRNA sequencing. PLoS Biol. 6, e280.
Edgar, R. C. (2010). Search and clustering orders of magnitude faster than BLAST. Bioinforma. Oxf. Engl. 26, 2460-2461.
Falagas, M. E., Mavroudis, A. D., and Vardakas, K. Z. (2016). The antibiotic pipeline for multi-drug resistant gram negative bacteria: what can we expect? Expert Rev. Anti Infect. Ther. 14, 747-763.
Francino, M. P. (2016). Antibiotics and the Human Gut Microbiome: Dysbioses and Accumulation of Resistances. Front. Microbiol. 6.
Ganz, T. (2003). The Role of Antimicrobial Peptides in Innate Immunity. Integr. Comp. Biol. 43, 300-304.
Hilton, D. J., Watowich, S. S., Katz, L., and Lodish, H. F. (1996). Saturation mutagenesis of the WSXWS motif of the erythropoietin receptor. J. Biol. Chem. 271, 4699-4708.
Jackson, M. A., Verdi, S., Maxan, M.-E., Shin, C. M., Zierer, J., Bowyer, R. C. E., Martin, T., Williams, F. M. K., Menni, C., Bell, J. T., et al. (2018). Gut microbiota associations with common diseases and prescription medications in a population-based cohort. Nat. Commun. 9,2655.
Kelly, R., and Davies, S.C. (2017). Tackling antimicrobial resistance globally. Med. J. Aust. 207, 371-373.
Kim, S., Covington, A., and Pamer, E. G. (2017). The intestinal microbiota: Antibiotics, colonization resistance, and enteric pathogens. Immunol. Rev. 279,90-105.
Lange, K., Buerger, M., Stallmach, A., and Bruns, T. (2016). Effects of Antibiotics on Gut Microbiota. Dig. Dis. Basel Switz. 34, 260-268.
de Lastours, V., and Fantin, B. (2015). Impact of fluoroquinolones on human microbiota. Focus on the emergence of antibiotic resistance. Future Microbiol. 10, 1241-1255.
Lowman, H. B., Bass, S. H., Simpson, N., and Wells, J. A. (1991). Selecting high-affinity binding proteins by monovalent phage display. Biochemistry 30, 10832-10838.
Maier, L., Goemans, C. V., Pruteanu, M., Wirbel, J., Kuhn, M., Cacace, E., Banerjee, T., Anderson, E. E., Milanese, A., Löber, U., et al. (2020). Dissecting the collateral damage of antibiotics on gut microbes. BioRxiv 2020.01.09.893560.
Needleman, S. B., and Wunsch, C. D. (1970). A general method applicable to the search for similarities in the amino acid sequence of two proteins. J. Mol. Biol. 48, 443-453.
Ner, S. S., Goodin, D. B., and Smith, M. (1988). A simple and efficient procedure for generating random point mutations and for codon replacements using mixed oligodeoxynucleotides. DNA Mary Ann Liebert Inc 7, 127-134.
Peschel, A., and Collins, L. V. (2001). Staphylococcal resistance to antimicrobial peptides of mammalian and bacterial origin. Peptides 22, 1651-1659.
Peschel, A., and Sahl, H.-G. (2006). The co-evolution of host cationic antimicrobial peptides and microbial resistance. Nat. Rev. Microbiol. 4, 529-536.
Raschig, J., Mailänder-Sánchez, D., Berscheid, A., Berger, J., Strömstedt, A. A., Courth, L. F., Malek, N. P., Brätz-Oesterhelt, H., and Wehkamp, J. (2017). Ubiquitously expressed Human Beta Defensin 1 (hBD1) forms bacteria-entrapping nets in a redox dependent mode of action. PLoS Pathog. 13, e1006261.
Reidhaar-Olson, J. F., and Sauer, R. T. (1988). Combinatorial cassette mutagenesis as a probe of the informational content of protein sequences. Science 241, 53-57.
Rice, P., Longden, I., and Bleasby, A. (2000). EMBOSS: the European Molecular Biology Open Software Suite. Trends Genet. TIG 16, 276-277.
Schroeder, B. O., Wu, Z., Nuding, S., Groscurth, S., Marcinowski, M., Beisner, J., Buchner, J., Schaller, M., Stange, E. F., and Wehkamp, J. (2011). Reduction of disulphide bonds unmasks potent antimicrobial activity of human β-defensin 1. Nature 469, 419-423.
Stehr, M., Greweling, M. C., Tischer, S., Singh, M., Blöcker, H., Monner, D. A., and Müller, W. (2009). Charles River altered Schaedler flora (CRASF) remained stable for four years in a mouse colony housed in individually ventilated cages. Lab. Anim. 43, 362-370.
Tacconelli, E., Carrara, E., Savoldi, A., Harbarth, S., Mendelson, M., Monnet, D. L., Pulcini, C., Kahlmeter, G., Kluytmans, J., Carmeli, Y., et al. (2018). Discovery, research, and development of new antibiotics: the WHO priority list of antibiotic-resistant bacteria and tuberculosis. Lancet Infect. Dis. 18, 318-327.
Thaiss, C. A., Zmora, N., Levy, M., and Elinav, E. (2016). The microbiome and innate immunity. Nature 535, 65-74.
The Lancet, null (2020). The antimicrobial crisis: enough advocacy, more action. Lancet Lond. Engl. 395, 247.
Thiemann, S., Smit, N., Roy, U., Lesker, T. R., Gálvez, E. J. C., Helmecke, J., Basic, M., Bleich, A., Goodman, A. L., Kalinke, U., et al. (2017). Enhancement of IFNγ Production by Distinct Commensals Ameliorates Salmonella-Induced Disease. Cell Host Microbe 21, 682-694.e5.
Wanner, S., Schade, J., Keinhörster, D., Weller, N., George, S. E., Kull, L., Bauer, J., Grau, T., Winstel, V., Stoy, H., et al. (2017). Wall teichoic acids mediate increased virulence in Staphylococcus aureus. Nat. Microbiol. 2, 16257.
Weidenmaier, C., Peschel, A., Xiong, Y.-Q., Kristian, S. A., Dietz, K., Yeaman, M. R., and Bayer, A. S. (2005). Lack of wall teichoic acids in Staphylococcus aureus leads to reduced interactions with endothelial cells and to attenuated virulence in a rabbit model of endocarditis. J. Infect. Dis. 191, 1771-1777.
Wendler, J., Schroeder, B. O., Ehmann, D., Koeninger, L., Mailänder-Sánchez, D., Lemberg, C., Wanner, S., Schaller, M., Stange, E. F., Malek, N. P., et al. (2019). Proteolytic Degradation of reduced Human Beta Defensin 1 generates a Novel Antibiotic Octapeptide. Sci. Rep. 9, 3640.
Wiles, S., Clare, S., Harker, J., Huett, A., Young, D., Dougan, G., and Frankel, G. (2004). Organ specificity, colonization and clearance dynamics in vivo following oral challenges with the murine pathogen Citrobacter rodentium. Cell. Microbiol. 6, 963-972.
Zasloff, M. (2002). Antimicrobial peptides of multicellular organisms. Nature 415, 389-395.

### Items

1. A compound having the structure a-b-c or c-b-a, wherein a) is a fatty moiety; b) is an optional linker/spacer and c) is a peptide selected from any α- or β-defensin fragment e.g. fragments of Human Defensin 5 (HD5); fragments of human neutrophil defensin 4 (HNP4) and/or human beta defensin-1 (hBD-1).
2. A compound having the structure a-b-c, wherein a) is a fatty moiety selected from C₄-C₂₇ long chain fatty acids such as butyric acid, lauric acid, palmitic acid or cholesterol, b) is an optional linker/spacer and/or PEG agents and c) is a peptide selected from any α- or β-defensin fragment e.g. fragments of Human Defensin 5 (HD5); fragments of human neutrophil defensin 4 (HNP4) and/or human beta defensin-1 (hBD-1).
3. A compound having the structure a-b-c, wherein a) is a fatty moiety; b) is an optional linker/spacer selected from sugars and/or amino acids and/or PEG agents and c) is a peptide selected from any α- or β-defensin fragment e.g. fragments of Human Defensin 5 (HD5); fragments of human neutrophil defensin 4 (HNP4) and/or human beta defensin-1 (hBD-1).
4. A compound having the structure a-b-c, wherein a) is palmitic acid; b) is an optional linker/spacer selected from sugars and/or amino acids and/or PEG agents and c) is a peptide selected from any α- or β-defensin fragment e.g. fragments of Human Defensin 5 (HD5); fragments of human neutrophil defensin 4 (HNP4) and/or human beta defensin-1 (hBD-1).
5. A compound having the structure a-b-c, wherein a) is palmitic acid; b) is 8-amino-3.6-dioxaoctanoic acid, a hydrophilic PEG agent and c) is a peptide selected from any α- or β-defensin fragment e.g. fragments of Human Defensin 5 (HD5); fragments of human neutrophil defensin 4 (HNP4) and/or human beta defensin-1 (hBD-1).
6. The chemical modification of any α- or β-defensin fragment e.g. fragments of Human Defensin 5 (HD5); fragments of human neutrophil defensin 4 (HNP4) and/or human beta defensin-1 (hBD-1) with palmitic acid and/or 8-amino-3.6-dioxaoctanoic acid to preserve and/or increase microbiota e.g. bacterial abundance, gene richness and/or bacterial phylae.
7. The chemical modification of any α- or β-defensin fragment e.g. fragments of Human Defensin 5 (HD5); fragments of human neutrophil defensin 4 (HNP4) and/or human beta defensin-1 (hBD-1) with palmitic acid and/or 8-amino-3.6-dioxaoctanoic acid to limit toxicity.
8. The chemical modification of any α- or β-defensin fragment e.g. fragments of Human Defensin 5 (HD5); fragments of human neutrophil defensin 4 (HNP4) and/or human beta defensin-1 (hBD-1) with palmitic acid and/or 8-amino-3.6-dioxaoctanoic acid to decrease and/or slow development of bacterial resistance.
9. A peptide having antimicrobial activity, which peptide is a modified fragment of human beta defensin-1 (hBD-1), wherein the peptide consists of the sequence of:
   Pam2-Glc-Suc-RGKAKCCK (PAM-1)
   Pam-RGKAKCCK (PAM-2),
   Pam-Ado-RGKAKCCK (PAM-3),
   Pam3Cys-RGKAKCCK (PAM-4),
   Pam-Lys(Pam)-RGKAKCCK (PAM-5).
10. The peptide, compound, or chemical modification of items 1 to 8, wherein the peptide consists of the sequence of:
   Pam-RGKAKCCK (PAM-2),
   Pam-Ado-RGKAKKC (PAM-3).
11. The peptide, compound, or chemical modification of items 1 to 8, wherein the peptide consists of the sequence of:
   Pam-Ado-RGKAKCCK (PAM-3).
12. The peptide, compound, or chemical modification of any of the preceding items, wherein the peptide is the C-terminal eight amino acids of hBD-1 chemically modified with palmitic acid (PAM-2) and/or 8-amino-3.6-dioxaoctanoic acid (PAM-3).
13. The peptide, compound, or chemical modification of any of items 1 to 12 for use in the treatment of any Gram-positive and/or Gram-negative bacterial infection, viral infections, protozoal infections, fungal infections or worm infections (helminthiasis).
14. The peptide, compound, or chemical modification of any of items 1 to 12 for use in the treatment of any drug resistant Gram-positive and/or Gram-negative bacterial infection.
15. The peptide, compound, or chemical modification of any of items 1 to 12 for use in the treatment of drug resistant viral infections.
16. The peptide, compound, or chemical modification of any of items 1 to 12 for use in the treatment of drug resistant protozoal infections.

17. The peptide, compound, or chemical modification of any of items 1 to 12 for use in the treatment of drug resistant fungal infections.
18. The peptide, compound, or chemical modification of any of items 1 to 12 for use in the treatment of drug resistant worm infections (helminthiasis).

19. The peptide, compound, or chemical modification of any of items 1 to 12 for use in the treatment of any bacterial infection caused by ESKAPE pathogens (E. faecium; S. aureus; K. pneumoniae; A. baumanii; P. aeruginosa and/or Enterobacter).
20. The peptide, compound, or chemical modification of any of items 1 to 12 for use in the treatment of any Gram-positive and/or Gram-negative bacterial gastrointestinal infection.
21. The peptide, compound, or chemical modification of any of items 1 to 12 for use in the treatment of any drug resistant Gram-positive and/or Gram-negative bacterial gastrointestinal infection.
22. The peptide, compound, or chemical modification of any of items 1 to 12 for use in the treatment of any gastrointestinal infection caused by ESKAPE pathogens (E. faecium; S. aureus; K. pneumoniae; A. baumanii; P. aeruginosa and/or Enterobacter).
23. The peptide, compound, or chemical modification of any of items 1 to 12 for use in the treatment of any bacterial infection specifically killing the pathogens through biofilm eradication and bacterial membrane disruption while preserving or increasing the microbiota e.g. bacterial abundance, richness and diversity and/or bacterial phylae with minimal development of bacterial resistance.
24. The peptide, compound, or chemical modification of any of items 1 to 12 for use in the treatment of any bacterial, gastrointestinal infection specifically killing the pathogens through biofilm eradication and bacterial membrane disruption while preserving or increasing the microbiota e.g. bacterial abundance, richness and diversity and/or bacterial phylae with minimal development of bacterial resistance.
25. The peptide, compound, or chemical modification of any of items 1 to 12 for use in the treatment of any bacterial infection specifically killing the pathogens while preserving or increasing the microbiota e.g. bacterial gene richness and/or bacterial phylae.
26. The peptide, compound, or chemical modification of any of items 1 to 12 for use in the treatment of any bacterial, gastrointestinal infection killing the pathogens while preserving or increasing the gastrointestinal microbiota e.g. bacterial gene richness and/or bacterial phylae.
27. The peptide, compound, or chemical modification of any of items 1 to 12 for use in the killing of any pathogenic bacteria through biofilm eradication and bacterial membrane disruption.
28. The peptide, compound, or chemical modification of any of items 1 to 12 for use in the killing of any pathogenic, gastrointestinal bacteria through biofilm eradication and bacterial membrane disruption.
29. The peptide, compound, or chemical modification of any items 1 to 12 for use in the treatment of any bacterial, gastrointestinal infection preserving the gastrointestinal microbiota thus decreasing the risk of secondary gastrointestinal infections with e.g. C. difficile.
30. The peptide, compound, or chemical modification of any of items 1 to 12 for use in the treatment of any bacterial infection caused by S. aureus; E. coli; C. rodentium; P. aeruginosa; S. typhimurium and/or S. enteritidis.
31. The peptide, compound, or chemical modification of any of items 1 to 12 for use in the treatment of any bacterial, gastrointestinal infection caused by S. aureus; E. coli; C. rodentium; P. aeruginosa S. enteritidis and/or S. typhimurium.
32. The peptide, compound, or chemical modification of any of items 1 to 12 for use in the treatment of any bacterial infection caused by S. aureus.
33. The peptide, compound, or chemical modification of any of items 1 to 12 for use in the treatment of any bacterial, gastrointestinal infection caused by S. aureus.
34. The peptide, compound, or chemical modification of any of items 1 to 12 for use in the treatment of any bacterial infection caused by E. coli.
35. The peptide, compound, or chemical modification of items 1 to 12 for use in the treatment of any bacterial, gastrointestinal infection caused by E. coli.
36. The peptide, compound, or chemical modification of any of items 1 to 12 for use in the treatment of any bacterial infection caused by C. rodentium.
37. The peptide, compound, or chemical modification of any of items 1 to 12 for use in the treatment of any bacterial, gastrointestinal infection caused by C. rodentium.
38. The peptide, compound, or chemical modification of any of items 1 to 12 for use in the treatment of any bacterial infection caused by P. aeruginosa.
39. The peptide, compound, or chemical modification of any of items 1 to 12 for use in the treatment of any bacterial, gastrointestinal infection caused by P. aeruginosa.
40. The peptide, compound, or chemical modification of any of items 1 to 12 for use in the treatment of any bacterial infection caused by S. enteritidis.
41. The peptide, compound, or chemical modification of any of items 1 to 12 for use in the treatment of any bacterial, gastrointestinal infection caused by S. enteritidis.
42. The peptide, compound, or chemical modification of any of items 1 to 12 for use in the treatment of any bacterial infection caused by S. typhimurium.
43. The peptide, compound, or chemical modification of any of items 1 to 12 for use in the treatment of any bacterial, gastrointestinal infection caused by S. typhimurium.
44. The peptide, compound, or chemical modification of any of items 1 to 12 for use in the treatment of any fungal infection caused by C. albicans.
45. The peptide, compound, or chemical modification of any of items 1 to 12 for use in the treatment of any fungal, gastrointestinal infection caused by C. albicans.
46. The treatments or uses described in any of the preceding items where the treatment consists of treatment with Pam2-Glc-Suc-RGKAKCCK (PAM-1) and/or Pam-RGKAKCCK (PAM-2) and/or Pam-Ado-RGKAKCCK (PAM-3) and/or Pam3Cys-RGKAKCCK (PAM-4) and/or Pam-Lys(Pam)-RGKAKCCK (PAM-5).
47. The treatments, or uses described in any of the preceding items where the treatment consists of treatment with Pam-RGKAKCCK (PAM-2) and/or Pam-Ado-RGKAKCCK (PAM-3).
48. The treatments or uses described in any of the preceding items where the treatment consists of treatment with Pam-Ado-RGKAKCCK (PAM-3).
49. The method, treatment, or use according to any of the preceding items, wherein PAM-1 and/or PAM-2 and/or PAM-3 and/or PAM-4 and/or PAM-5 is administered to a subject in need thereof at a daily dose of between 1 mg/kg and 1000 mg/kg.
50. The method, treatment, or use according to any one of the preceding items, wherein PAM-1 and/or PAM-2 and/or PAM-3 and/or PAM-4 and/or PAM-5 is administered to a subject in need thereof two times a day.
51. The method, treatment, or use according to any of the preceding items, wherein PAM-1 and/or PAM-2 and/or PAM-3 and/or PAM-4 and/or PAM-5 is administered to a subject in need thereof once daily.
52. The method, treatment, or use according to any of the preceding items, wherein PAM-3 is administered to a subject in need thereof two times a day or continuously.
53. The method, treatment, or use according to any of the preceding items, wherein the administration of PAM-1 and/or PAM-2 and/or PAM-3 and/or PAM-4 and/or PAM-5 is oral, topical (i.e. eye, ear, nose, skin), intravaginal, rectal, intrathecal, intrapulmonary or intravenous.
54. The method, treatment, or use according to any of the preceding items, wherein PAM-3 is administered orally.
55. The method, treatment, or use according to any of the preceding items, wherein PAM-3 is administered intrapulmonary.
56. A medicament comprising a peptide of any of items 1-12 and a pharmaceutically acceptable carrier.

1. A compound having the structure a-b-c, wherein a) comprises or is lauric acid, palmitic acid, or cholesterol; b) is an optional linker/spacer selected from sugars and/or amino acids and/or PEG agents and c) is a peptide selected from fragments of human beta defensin-1 (hBD-1).

2. The compound of claim 1, wherein a comprises or is lauric or palmitic acid, preferably palmitic acid.

3. The compound of claim 1 or 2, wherein the fragment of hBD-1 is SEQ ID NO: 36 or 39.

4. The compound of any of the preceding claims, wherein the compound comprises or consists of the C-terminal eight amino acids of hBD-1 chemically modified with palmitic acid and/or 8-amino-3.6-dioxaoctanoic acid.

5. The compound of any of the preceding claims, wherein the compound has antimicrobial activity.

6. The compound of any of the preceding claims, wherein the compound is a modified fragment of human beta defensin-1 (hBD-1), wherein the compound is selected from the list:
Pam2-Glc-Suc-RGKAKCCK (PAM-1)
Pam-RGKAKCCK (PAM-2),
Pam-Ado-RGKAKCCK (PAM-3),
Pam3Cys-RGKAKCCK (PAM-4), and
Pam-Lys(Pam)-RGKAKCCK (PAM-5).

7. The compound of any of the preceding claims for use in the treatment of any Gram-positive and/or Gram-negative bacterial infection, viral infections, protozoal infections, fungal infections or worm infections (helminthiasis).

8. The compound of any of the preceding claims for use in the treatment of any drug resistant Gram-positive and/or Gram-negative bacterial infection.

9. The compound of any of the preceding claims for use in the treatment of drug resistant viral infections.

10. The compound of any of the preceding claims for use in the treatment of drug resistant protozoal infections.

11. The compound of any of the preceding claims for use in the treatment of drug resistant fungal infections.

12. The compound of any of the preceding claims for use in the treatment of drug resistant worm infections (helminthiasis).

13. The compound of any of the preceding claims for use in the treatment of any bacterial infection caused by ESKAPE pathogens (E. faecium; S. aureus; K. pneumoniae; A. baumanii; P. aeruginosa and/or Enterobacter).

14. The compound of any of the preceding claims for use in the treatment of any Gram-positive and/or Gram-negative bacterial gastrointestinal infection.

15. The compound of any of the preceding claims for use in the treatment of any drug resistant Gram-positive and/or Gram-negative bacterial gastrointestinal infection.

16. The compound of any of the preceding claims for use in the treatment of any gastrointestinal infection caused by ESKAPE pathogens (E. faecium; S. aureus; K. pneumoniae; A. baumanii; P. aeruginosa and/or Enterobacter).

17. The compound of any of the preceding claims for use in the treatment of any bacterial infection specifically killing the pathogens through biofilm eradication and bacterial membrane disruption while preserving or increasing the microbiota e.g. bacterial abundance, richness and diversity and/or bacterial phylae with minimal development of bacterial resistance.

18. The compound of any of the preceding claims for use in the treatment of any bacterial, gastrointestinal infection specifically killing the pathogens through biofilm eradication and bacterial membrane disruption while preserving or increasing the microbiota e.g. bacterial abundance, richness and diversity and/or bacterial phylae with minimal development of bacterial resistance.

19. The compound of any of the preceding claims for use in the treatment of any bacterial infection specifically killing the pathogens while preserving or increasing the microbiota e.g. bacterial gene richness and/or bacterial phylae.

20. The compound of any of the preceding claims for use in the treatment of any bacterial, gastrointestinal infection killing the pathogens while preserving or increasing the gastrointestinal microbiota e.g. bacterial gene richness and/or bacterial phylae.

21. The compound of any of the preceding claims for use in the killing of any pathogenic bacteria through biofilm eradication and bacterial membrane disruption.

22. The compound of any of the preceding claims for use in the killing of any pathogenic, gastrointestinal bacteria through biofilm eradication and bacterial membrane disruption.

23. The compound of any the preceding claims for use in the treatment of any bacterial, gastrointestinal infection preserving the gastrointestinal microbiota thus decreasing the risk of secondary gastrointestinal infections with e.g. C. difficile.

24. The compound of any of the preceding claims for use in the treatment of any bacterial infection caused by S. aureus; E. coli; C. rodentium; P. aeruginosa; S. typhimurium and/or S. enteritidis.

25. The compound of any of the preceding claims for use in the treatment of any bacterial, gastrointestinal infection caused by S. aureus; E. coli; C. rodentium; P. aeruginosa S. enteritidis and/or S. typhimurium.

26. The compound of any of the preceding claims for use in the treatment of any bacterial infection caused by S. aureus.

27. The compound of any of the preceding claims for use in the treatment of any bacterial, gastrointestinal infection caused by S. aureus.

28. The compound of any of the preceding claims for use in the treatment of any bacterial infection caused by E. coli.

29. The compound of the preceding claims for use in the treatment of any bacterial, gastrointestinal infection caused by E. coli.

30. The compound of any of the preceding claims for use in the treatment of any bacterial infection caused by C. rodentium.

31. The compound of any of the preceding claims for use in the treatment of any bacterial, gastrointestinal infection caused by C. rodentium.

32. The compound of any of the preceding claims for use in the treatment of any bacterial infection caused by P. aeruginosa.

33. The compound of any of the preceding claims for use in the treatment of any bacterial, gastrointestinal infection caused by P. aeruginosa.

34. The compound of any of the preceding claims for use in the treatment of any bacterial infection caused by S. enteritidis.

35. The compound of any of the preceding claims for use in the treatment of any bacterial, gastrointestinal infection caused by S. enteritidis.

36. The compound of any of the preceding claims for use in the treatment of any bacterial infection caused by S. typhimurium.

37. The compound of any of the preceding claims for use in the treatment of any bacterial, gastrointestinal infection caused by S. typhimurium.

38. The compound of any of the preceding claims for use in the treatment of any fungal infection caused by C. albicans.

39. The compound of any of the preceding claims for use in the treatment of any fungal, gastrointestinal infection caused by C. albicans.
